(19) United States
(12) Patent Application Publication
(10) Pub. No.: US 2025/0012785 A1
(43) Pub. Date: Jan. 9, 2025

(54) SYSTEMS AND METHODS FOR DETECTION AND QUANTIFICATION OF DOUBLE-STRANDED RNA

(71) Applicant: Promega Corporation, Madison, WI (US)

(72) Inventors: Jamison GRAILER, Madison, WI (US); Richard A. Moravec, Madison, WI (US); James R. Hartnett, Madison, WI (US); Mei Cong, Madison, WI (US)

(21) Appl. No.: 18/735,719

(22) Filed: Jun. 6, 2024

Related U.S. Application Data
(60) Provisional application No. 63/506,502, filed on Jun. 6, 2023.

(51) Int. Cl. G01N 33/53; C07K 14/435; C12N 9/02; C12N 9/12

(52) U.S. Cl. CPC G01N 33/5308; C07K 14/43595; C12N 9/0069; C12N 9/12; C12Y 113/12013; C12Y 207/11001; C07K 2319/60; C07K 2319/61; G01N 2470/06

(57) ABSTRACT

Provided herein are compositions comprising a double-stranded RNA (dsRNA) binding domains linked to components of a complementation system. Upon binding of a pair of dsRNA binding domains to a dsRNA, a detectable complex of the complementation components is formed, and the dsRNA can be detected/quantified.

### CROSS-REFERENCE TO RELATED APPLICATIONS

The present invention claims the priority benefit of U.S. Provisional Patent Application 63/506,502, filed Jun. 6, 2023, which is incorporated by reference in its entirety.

### SEQUENCE LISTING

The text of the computer readable sequence listing filed herewith titled “PRMG_41970_202_SequenceListing.xml,” created Jun. 5, 2024, having a file size of 3,253,101 bytes, is hereby incorporated by reference in its entirety.

### FIELD

Provided herein are compositions comprising a double-stranded RNA (dsRNA) binding domains linked to components of a complementation system. Upon binding of a pair of dsRNA binding domains to a dsRNA, a detectable complex of the complementation components is formed, and the dsRNA can be detected/quantified.

### BACKGROUND

Double-stranded RNA (dsRNA) is a byproduct and contaminant of in vitro mRNA transcription during, for example, production of mRNA-based therapeutics (vaccines, gene therapy). The FDA has issued a guidance document specifically focused on the development of RNA-based therapeutics, titled “Chemistry, Manufacturing, and Control (CMC) Information for Human Gene Therapy Investigational New Drug Applications (INDs)”. This guidance document provides recommendations for the development and characterization of RNA-based therapeutics, including considerations for the presence of dsRNA. Current guidance states that dsRNA needs to be measured for all in vitro transcribed RNA products. There is currently not a stated limit for the amount of dsRNA in RNA drug products.

Currently, quantitation of dsRNA (i.e., specific for dsRNA, not ssRNA or DNA) can be performed by two accepted methods, ELISA and dot blot. Enzyme-linked immunosorbent assay (ELISA) can be used to quantitate dsRNA using specific antibodies that recognize dsRNA. Sensitivity may be an issue as commercially available ELISA kits exhibit a sensitivity of 2-5 ng/ml. The accepted ELISA Ab clones are J2, K1, K2. However, despite being the current “gold standard” for dsRNA detection, there are recognized problems with these clones in the field. For example, clone J2 exhibits preferred binding to the ends of dsRNA oligos as well as an internal binding site of A2N9A3N9A2 (where adenine is presented on one face of the helix, see Bonin et al., RNA, 2000). Therefore, this antibody is not sequence agnostic and does not represent accurate quantitation of dsRNA. The other clone, K1, displays altered binding kinetics when testing dsRNA from different sources. Therefore, these clones do not provide true quantitation of dsRNA. Dot blots can be used for dsRNA detection although they are not as sensitive or quantitative as ELISA, and they rely on the same flawed antibody clones.

What is needed is a quantitative, sensitive (e.g., limit of detection <1 ng/ml), specific, rapid (<2 hours assay time), and easy to use (add-mix-read) assay for quantitating dsRNA.

### SUMMARY

Provided herein are compositions comprising a double-stranded RNA (dsRNA) binding domains linked to components of a complementation system. Upon binding of a pair of dsRNA binding domains to a dsRNA, a detectable complex of the complementation components is formed, and the dsRNA can be detected/quantified.

In some embodiments, provided herein are double-stranded RNA (dsRNA) detection systems comprising: (a) a first fusion of (i) a first dsRNA binding domain and (ii) a first component of a detectable complex; and (b) a second fusion of (i) a second dsRNA binding domain and (ii) a second component of the detectable complex. In some embodiments, upon binding of the first and second dsRNA binding domains to a dsRNA, the first and second components of the detectable complex associate to form the detectable complex. In some embodiments, the first and second components of the detectable complex exhibit low affinity for one another in the absence of facilitation through the binding of the first and second dsRNA binding domains to the dsRNA.

In some embodiments, the first dsRNA binding domain and the second dsRNA binding domain comprise different amino acid sequences. In some embodiments, the first dsRNA binding domain and the second dsRNA binding domain comprise the same amino acid sequences. In some embodiments, a dsRNA binding domain comprises a dsRNA binding motif having at least 70% (e.g., 70%, 75%, 80%, 85%, 90%, 95%, 100%, or ranges therebetween) sequence similarity with SEQ ID NO: 3062 and/or SEQ ID NO: 3063. In some embodiments, a dsRNA binding domain comprises a dsRNA binding motif having at least 70% (e.g., 70%, 75%, 80%, 85%, 90%, 95%, 100%, or ranges therebetween) sequence identity with SEQ ID NO: 3062 and/or SEQ ID NO: 3063. In some embodiments, a dsRNA binding domain comprises a dsRNA binding motif of SEQ ID NO: 3062. In some embodiments, a dsRNA binding domain comprises a dsRNA binding motif of SEQ ID NO: 3063. In some embodiments, a dsRNA binding domain comprises dsRNA binding motifs having at least 70% (e.g., 70%, 75%, 80%, 85%, 90%, 95%, 100%, or ranges therebetween) sequence similarity with SEQ ID NO: 3062 and SEQ ID NO: 3063. In some embodiments, a dsRNA binding domain comprises dsRNA binding motifs having at least 70% (e.g., 70%, 75%, 80%, 85%, 90%, 95%, 100%, or ranges therebetween) sequence identity with SEQ ID NO: 3062 and SEQ ID NO: 3063. In some embodiments, a dsRNA binding domain comprises dsRNA binding motifs of SEQ ID NO: 3062 and SEQ ID NO: 3063. In some embodiments, a dsRNA binding domain comprises at least 70% (e.g., 70%, 75%, 80%, 85%, 90%, 95%, 100%, or ranges therebetween) sequence similarity with SEQ ID NO: 3061. In some embodiments, a dsRNA binding domain comprises at least 70% (e.g., 70%, 75%, 80%, 85%, 90%, 95%, 100%, or ranges therebetween) sequence identity with SEQ ID NO: 3061. In some embodiments, a dsRNA binding domain comprises SEQ ID NO: 3061.

In some embodiments, the detectable complex is capable of generating a signal that can be detected. In some embodiments, the amount of signal generated by the detectable complex can be correlated to the amount of dsRNA in a sample with the system. In some embodiments, the signal comprises one or more of fluorescence, luminescence, enzymatic activity, and ligand binding. In some embodiments, the first and second components of the detectable complex are fragments of a protein capable of generating a detectable signal, and wherein the detectable complex is capable of generating the detectable signal upon association of the first and second components of the detectable complex.

In some embodiments, the detectable signal is fluorescence. In some embodiments, the first and second components of the detectable complex comprise at least 70% (e.g., 70%, 75%, 80%, 85%, 90%, 95%, 100%, or ranges therebetween) sequence identity with first and second fragments of a fluorescent protein. In some embodiments, the fluorescent protein is selected from yellow fluorescent protein (YFP), green fluorescent protein (GFP), cyan fluorescent protein (CFP), red fluorescent protein (RFP), umbelliferone, fluorescein, fluorescein isothiocyanate, rhodamine, dichlorotriazinylamine fluorescein, cyanines, dansyl chloride, phycocyanin, and phycoerythrin.

In some embodiments, the detectable signal is enzymatic activity. In some embodiments, the first and second components of the detectable complex comprise at least 70% (e.g., 70%, 75%, 80%, 85%, 90%, 95%, 100%, or ranges therebetween) sequence identity with first and second fragments of an enzyme. In some embodiments, the enzyme is selected from beta-lactamase, dihydrofolate reductase (DHFR), focal adhesion kinase (FAK), Gal4, and horseradish peroxidase. In some embodiments, the detectable signal is luminescence in the presence of a substrate. In some embodiments, the first and second components of the detectable complex comprise at least 70% (e.g., 70%, 75%, 80%, 85%, 90%, 95%, 100%, or ranges therebetween) sequence identity with first and second fragments of an luciferase. In some embodiments, the luciferase is selected from an Oplophorus luciferase, a firefly luciferase, a click beetle luciferase, a Renilla luciferase, cypridina luciferase, an Aequorin photoprotein, and an obelin photoprotein. In some embodiments, the first and second components of the detectable complex collectively comprise at least 70% (e.g., 70%, 75%, 80%, 85%, 90%, 95%, 100%, or ranges therebetween) sequence identity with SEQ ID NO: 3041. In some embodiments, the first component of the detectable complex comprises at least 70% (e.g., 70%, 75%, 80%, 85%, 90%, 95%, 100%, or ranges therebetween) sequence identity with SEQ ID NO: 3042 and the first component of the detectable complex comprises at least 70% (e.g., 70%, 75%, 80%, 85%, 90%, 95%, 100%, or ranges therebetween) sequence identity with SEQ ID NO: 3050. In some embodiments, the first component of the detectable complex comprises at least 70% (e.g., 70%, 75%, 80%, 85%, 90%, 95%, 100%, or ranges therebetween) sequence identity with SEQ ID NO: 3043 and the first component of the detectable complex comprises at least 70% (e.g., 70%, 75%, 80%, 85%, 90%, 95%, 100%, or ranges therebetween) sequence identity with SEQ ID NO: 3051. In some embodiments, the first component of the detectable complex comprises at least 70% (e.g., 70%, 75%, 80%, 85%, 90%, 95%, 100%, or ranges therebetween) sequence identity with SEQ ID NO: 3044 and the first component of the detectable complex comprises at least 70% (e.g., 70%, 75%, 80%, 85%, 90%, 95%, 100%, or ranges therebetween) sequence identity with SEQ ID NO: 3052. In some embodiments, the first component of the detectable complex comprises at least 70% (e.g., 70%, 75%, 80%, 85%, 90%, 95%, 100%, or ranges therebetween) sequence identity with SEQ ID NO: 3045 and the first component of the detectable complex comprises at least 70% (e.g., 70%, 75%, 80%, 85%, 90%, 95%, 100%, or ranges therebetween) sequence identity with SEQ ID NO: 3053. In some embodiments, systems further comprise the substrate.

In some embodiments, the detectable signal is ligand binding. In some embodiments, the detectable complex is a modified dehalogenase complex and wherein the first and second components of the modified dehalogenase complex comprise at least 70% (e.g., 70%, 75%, 80%, 85%, 90%, 95%, 100%, or ranges therebetween) sequence identity with first and second fragments of a modified dehalogenase. In some embodiments, the modified dehalogenase comprises SEQ ID NO: 1. In some embodiments, systems further comprise a haloalkyl ligand for the modified dehalogenase. In some embodiments, the haloalkyl ligand comprises R-linker-A-X, wherein R is a detectable moiety, X is a halogen, and A-X is a substrate for a dehalogenase enzyme. In some embodiments, R is a fluorophore.

In some embodiments, provided herein are double-stranded RNA (dsRNA) detection systems comprising: (a) a first fusion of (i) a PKR-derived dsRNA binding domain sequence, and (ii) a peptide component of a bioluminescent complex; and (b) a second fusion of (i) the PKR-derived dsRNA binding domain sequence, and (ii) a polypeptide component of a bioluminescent complex; wherein upon binding of the PKR-derived dsRNA binding domain sequences to a dsRNA a luminescent complex is formed by structural complementation of the peptide component and the polypeptide component; and wherein a luminescent signal produced by the luminescent complex in the presence of the dsRNA and a substrate for the luminescent complex is enhanced when compared to a luminescent signal produced in the absence of the dsRNA. In some embodiments, systems further comprise the substrate for the luminescent complex. In some embodiments, the substrate for the luminescent complex is an imidazopyrazine luminophore. In some embodiments, the imidazopyrazine luminophore is coelenterazine or furimazine. In some embodiments, systems further comprise dsRNA. In some embodiments, the dsRNA binding domain comprises dsRNA binding motifs having at least 70% (e.g., 70%, 75%, 80%, 85%, 90%, 95%, 100%, or ranges therebetween) sequence similarity with SEQ ID NO: 3062 and SEQ ID NO: 3063. In some embodiments, the dsRNA binding domain comprises dsRNA binding motifs having at least 70% (e.g., 70%, 75%, 80%, 85%, 90%, 95%, 100%, or ranges therebetween) sequence identity with SEQ ID NO: 3062 and SEQ ID NO: 3063. In some embodiments, the dsRNA binding domain comprises at least 70% (e.g., 70%, 75%, 80%, 85%, 90%, 95%, 100%, or ranges therebetween) sequence similarity with SEQ ID NO: 3061. In some embodiments, the dsRNA binding domain comprises at least 70% (e.g., 70%, 75%, 80%, 85%, 90%, 95%, 100%, or ranges therebetween) sequence identity with SEQ ID NO: 3061. In some embodiments, the dsRNA binding domain comprises SEQ ID NO: 3061. In some embodiments, the peptide component has at least 70% (e.g., 70%, 75%, 80%, 85%, 90%, 95%, 100%, or ranges therebetween) sequence similarity with SEQ ID NO: 3038 and/or the polypeptide component has at least 70% (e.g., 70%, 75%, 80%, 85%, 90%, 95%, 100%, or ranges therebetween) sequence similarity with SEQ ID NO: 3037. In some embodiments, the peptide component has at least 70% (e.g., 70%, 75%, 80%, 85%, 90%, 95%, 100%, or ranges therebetween) sequence identity with SEQ ID NO: 3038 and/or the polypeptide component has at least 70% (e.g., 70%, 75%, 80%, 85%, 90%, 95%, 100%, or ranges therebetween) sequence identity with SEQ ID NO: 3037. In some embodiments, the peptide component comprises SEQ ID NO: 3038 and the polypeptide comprises SEQ ID NO: 3037.

In some embodiments, provided herein are methods of detecting dsRNA in a sample, the method comprising contacting the sample with a system described herein and detecting a signal from the detectable complex, wherein the amount of signal detected correlates with the amount of dsRNA in the sample. In some embodiments, the sample comprises a single stranded RNA-based therapeutic. In some embodiments, the sample further comprises a dsRNA (e.g., a contaminant).

### BRIEF DESCRIPTION OF THE DRAWINGS

FIG. 1. Cartoon depicting the binding of protein kinase R (PKR) to a dsRNA and dimerization of two PKR.

FIG. 2. Cartoon depicting an exemplary embodiment of the present technology in which two components of a luminescent complex (LgBiT and SmBIT) are provided as fusions with the dsRNA binding domain of PKR; binding of the dsRNA binding domains to a dsRNA results in formation of the active luminescent complex.

FIG. 3. Graph depicting increasing luminescent signal with increasing dsRNA in the presence of an exemplary system of FIG. 2.

FIG. 4. Graph depicting increasing fluorescent signal with increasing dsRNA in the presence of an exemplary system comprising fusions of a split GFP construct with the dsRNA binding domain of PKR (PKR-LgGFP and PKR-SmGFP).

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies, or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a,” “an,” and “the” include plural reference unless the context clearly dictates otherwise. Thus, for example, reference to “a polypeptide” is a reference to one or more polypeptides and equivalents thereof known to those skilled in the art, and so forth.

As used herein, the term “and/or” includes any and all combinations of listed items, including any of the listed items individually. For example, “A, B, and/or C” encompasses A, B, C, AB, AC, BC, and ABC, each of which is to be considered separately described by the statement “A, B, and/or C.”

As used herein, the term “comprise” and linguistic variations thereof denote the presence of recited feature(s), element(s), method step(s), etc., without the exclusion of the presence of additional feature(s), element(s), method step(s), etc. Conversely, the term “consisting of” and linguistic variations thereof, denotes the presence of recited feature(s), element(s), method step(s), etc., and excludes any unrecited feature(s), element(s), method step(s), etc., except for ordinarily-associated impurities. The phrase “consisting essentially of” denotes the recited feature(s), element(s), method step(s), etc., and any additional feature(s), element(s), method step(s), etc., that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

As used herein, the term “substantially” means that the recited characteristic, parameter, and/or value need not be achieved exactly, but that deviations or variations, including for example, tolerances, measurement error, measurement accuracy limitations and other factors known to skill in the art, may occur in amounts that do not preclude the effect the characteristic was intended to provide. A characteristic or feature that is substantially absent (e.g., substantially non-fluorescent) may be one that is within the noise, beneath background, below the detection capabilities of the assay being used, or a small fraction (e.g., <1%, <0.1%, <0.01%, <0.001%, <0.00001%, <0.000001%, <0.0000001%) of the significant characteristic (e.g., fluorescent intensity of an active fluorophore).

As used herein, when referring to amino acid sequences or positions within an amino acid sequence, the phrase “corresponding to” refers to the relative position of an amino acid residue or an amino acid segment with the sequence being referred to, not necessarily the specific identity of the amino acids at that position. For example, a “peptide corresponding to positions 36 through 48 of SEQ ID NO: 1” may comprise less than 100% sequence identity with positions 36 through 48 of SEQ ID NO: 1 (e.g., >70% sequence identity), but within the context of the composition or system being described the peptide relates to those positions.

As used herein, the term “system” refers to multiple components (e.g., devices, compositions, etc.) that find use for a particular purpose. For example, two separate biological molecules, whether present in the same composition or not, may comprise a system if they are useful together for a shared purpose.

As used herein, the term “complementary” refers to the characteristic of two or more structural elements (e.g., peptide, polypeptide, nucleic acid, small molecule, etc.) of being able to hybridize, dimerize, or otherwise form a complex with each other. For example, a “complementary peptide and polypeptide” are capable of coming together to form a complex. Complementary elements may require assistance (facilitation) to form a complex (e.g., from interaction elements), for example, to place the elements in the proper conformation for complementarity, to place the elements in the proper proximity for complementarity, to co-localize complementary elements, to lower interaction energy for complementary, to overcome insufficient affinity for one another, etc.

As used herein, the term “complex” refers to an assemblage or aggregate of molecules (e.g., peptides, polypeptides, etc.) in direct and/or indirect contact with one another. In one aspect, “contact,” or more particularly “direct contact,” means two or more molecules are close enough so that attractive noncovalent interactions, such as Van der Waal forces, hydrogen bonding, ionic and hydrophobic interactions, and the like, dominate the interaction of the molecules. In such an aspect, a complex of molecules (e.g., peptides, polypeptides, etc.) is formed under assay conditions such that the complex is thermodynamically favored (e.g., compared to a non-aggregated, or non-complexed, state of its component molecules). As used herein the term “complex,” unless described as otherwise, refers to the assemblage of two or more molecules (e.g., peptides, polypeptides, etc.). The molecules that assemble to for a complex are referred to herein as “components” or linguistic variations thereof.

As used herein, the term “low affinity” describes an intermolecular interaction between two or more entities that is too weak to result in significant complex formation between the entities, except at concentrations substantially higher (e.g., 2-fold, 5-fold, 10-fold, 100-fold, 1000-fold, or more) than physiologic or assay conditions, or with facilitation from the formation of a second complex of attached elements (e.g., interaction elements).

As used herein, the term “high affinity” describes an intermolecular interaction between two or more (e.g., three) entities that is of sufficient strength to produce detectable complex formation under physiologic or assay conditions without facilitation from the formation of a second complex of attached elements (e.g., interaction elements).

The term “amino acid” refers to natural amino acids, unnatural amino acids, and amino acid analogs, all in their D and L stereoisomers, unless otherwise indicated, if their structures allow such stereoisomeric forms.

The term “proteinogenic amino acids” refers to the 20 amino acids coded for in the human genetic code, and includes alanine (Ala or A), arginine (Arg or R), asparagine (Asn or N), aspartic acid (Asp or D), cysteine (Cys or C), glutamine (Gln or Q), glutamic acid (Glu or E), glycine (Gly or G), histidine (His or H), isoleucine (Ile or I), leucine (Leu or L), Lysine (Lys or K), methionine (Met or M), phenylalanine (Phe or F), proline (Pro or P), serine (Ser or S), threonine (Thr or T), tryptophan (Trp or W), tyrosine (Tyr or Y) and valine (Val or V). Selenocysteine and pyrrolysine may also be considered proteinogenic amino acids

The term “non-proteinogenic amino acid” refers to an amino acid that is not naturally-encoded or found in the genetic code of any organism, and is not incorporated biosynthetically into proteins during translation. Non-proteinogenic amino acids may be “unnatural amino acids” (amino acids that do not occur in nature) or “naturally-occurring non-proteinogenic amino acids” (e.g., norvaline, ornithine, homocysteine, etc.). Examples of non-proteinogenic amino acids include, but are not limited to, azetidinecarboxylic acid, 2-aminoadipic acid, 3-aminoadipic acid, beta-alanine, naphthylalanine, aminopropionic acid, 2-aminobutyric acid, 4-aminobutyric acid, 6-aminocaproic acid, 2-aminoheptanoic acid, 2-aminoisobutyric acid, 3-aminoisbutyric acid, 2-aminopimelic acid, tertiary-butylglycine, 2,4-diaminoisobutyric acid, desmosine, 2,2′-diaminopimelic acid, 2,3-diaminopropionic acid, N-ethylglycine, N-ethylasparagine, homoproline, hydroxylysine, allo-hydroxylysine, 3-hydroxyproline, 4-hydroxyproline, isodesmosine, allo-isoleucine, N-methylalanine, N-alkylglycine including N-methylglycine, N-methylisoleucine, N-alkylpentylglycine including N-methylpentylglycine, N-methylvaline, naphthylalanine, norvaline, norleucine (“Norleu”), octylglycine, ornithine, pentylglycine, pipecolic acid, thioproline, homolysine, and homoarginine. Non-proteinogenic also include D-amino acid forms of any of the amino acids herein, as well as non-alpha amino acid forms of any of the amino acids herein (beta-amino acids, gamma-amino acids, delta-amino acids, etc.), all of which are in the scope herein and may be included in peptides herein.

The term “amino acid analog” refers to an amino acid (e.g., natural or unnatural, proteinogenic or non-proteinogenic) where one or more of the C-terminal carboxy group, the N-terminal amino group and side-chain bioactive group has been chemically blocked, reversibly or irreversibly, or otherwise modified to another bioactive group. For example, aspartic acid-(beta-methyl ester) is an amino acid analog of aspartic acid; N-ethylglycine is an amino acid analog of glycine; or alanine carboxamide is an amino acid analog of alanine. Other amino acid analogs include methionine sulfoxide, methionine sulfone, S-(carboxymethyl)-cysteine, S-(carboxymethyl)-cysteine sulfoxide, and S-(carboxymethyl)-cysteine sulfone.

As used herein, unless otherwise specified, the terms “peptide” and “polypeptide” refer to polymer compounds of two or more amino acids joined through the main chain by peptide amide bonds (—C(O) NH—). The term “peptide” typically refers to short amino acid polymers (e.g., chains having fewer than 30 amino acids), whereas the term “polypeptide” typically refers to longer amino acid polymers (e.g., chains having more than 30 amino acids).

As used herein, a “conservative” amino acid substitution refers to the substitution of an amino acid in a peptide or polypeptide with another amino acid having similar chemical properties such as size or charge. For purposes of the present disclosure, each of the following eight groups contains amino acids that are conservative substitutions for one another:
1) Alanine (A) and Glycine (G);
2) Aspartic acid (D) and Glutamic acid (E);
3) Asparagine (N) and Glutamine (Q);
4) Arginine (R) and Lysine (K);
5) Isoleucine (I), Leucine (L), Methionine (M), and Valine (V);
6) Phenylalanine (F), Tyrosine (Y), and Tryptophan (W);
7) Serine(S) and Threonine (T); and
8) Cysteine (C) and Methionine (M).

Amino acid residues may be divided into classes based on common side chain properties, for example: polar positive (or basic) (e.g., histidine (H), lysine (K), and arginine (R)); polar negative (or acidic) (e.g., aspartic acid (D), glutamic acid (E)); polar neutral (e.g., serine(S), threonine (T), asparagine (N), glutamine (Q)); non-polar aliphatic (e.g., alanine (A), valine (V), leucine (L), isoleucine (I), methionine (M)); non-polar aromatic (e.g., phenylalanine (F), tyrosine (Y), tryptophan (W)); proline and glycine; and cysteine. As used herein, a “semi-conservative” amino acid substitution refers to the substitution of an amino acid in a peptide or polypeptide with another amino acid within the same class.

In some embodiments, unless otherwise specified, a conservative or semi-conservative amino acid substitution may also encompass non-naturally occurring amino acid residues that have similar chemical properties to the natural residue. These non-natural residues are typically incorporated by chemical peptide synthesis rather than by synthesis in biological systems. These include, but are not limited to, peptidomimetics and other reversed or inverted forms of amino acid moieties. Embodiments herein may, in some embodiments, be limited to natural amino acids, non-natural amino acids, and/or amino acid analogs.

Non-conservative substitutions may involve the exchange of a member of one class for a member from another class.

As used herein, the term “sequence identity” refers to the degree two polymer sequences (e.g., peptide, polypeptide, nucleic acid, etc.) have the same sequential composition of monomer subunits. The term “sequence similarity” refers to the degree with which two polymer sequences (e.g., peptide, polypeptide, nucleic acid, etc.) have similar polymer sequences. For example, similar amino acids are those that share the same biophysical characteristics and can be grouped into the families, e.g., acidic (e.g., aspartate, glutamate), basic (e.g., lysine, arginine, histidine), non-polar (e.g., alanine, valine, leucine, isoleucine, proline, phenylalanine, methionine, tryptophan) and uncharged polar (e.g., glycine, asparagine, glutamine, cysteine, serine, threonine, tyrosine). The “percent sequence identity” (or “percent sequence similarity”) is calculated by: (1) comparing two optimally aligned sequences over a window of comparison (e.g., the length of the longer sequence, the length of the shorter sequence, a specified window), (2) determining the number of positions containing identical (or similar) monomers (e.g., same amino acids occurs in both sequences, similar amino acid occurs in both sequences) to yield the number of matched positions, (3) dividing the number of matched positions by the total number of positions in the comparison window (e.g., the length of the longer sequence, the length of the shorter sequence, a specified window), and (4) multiplying the result by 100 to yield the percent sequence identity or percent sequence similarity. For example, if peptides A and B are both 20 amino acids in length and have identical amino acids at all but 1 position, then peptide A and peptide B have 95% sequence identity. If the amino acids at the non-identical position shared the same biophysical characteristics (e.g., both were acidic), then peptide A and peptide B would have 100% sequence similarity. As another example, if peptide C is 20 amino acids in length and peptide D is 15 amino acids in length, and 14 out of 15 amino acids in peptide D are identical to those of a portion of peptide C, then peptides C and D have 70% sequence identity, but peptide D has 93.3% sequence identity to an optimal comparison window of peptide C. For the purpose of calculating “percent sequence identity” (or “percent sequence similarity”) herein, any gaps in aligned sequences are treated as mismatches at that position.

Any peptide/polypeptides described herein as having a particular percent sequence identity or similarity (e.g., at least 70%) with a reference sequence ID number, may also be expressed as having a maximum number of substitutions (or terminal deletions) with respect to that reference sequence. For example, a sequence having at least Y % sequence identity (e.g., 90%) with SEQ ID NO:Z (e.g., 100 amino acids) may have up to X substitutions (e.g., 10) relative to SEQ ID NO:Z, and may therefore also be expressed as “having X (e.g., 10) or fewer substitutions relative to SEQ ID NO:Z.”

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum, and the like. Sample may also refer to cell lysates or purified forms of the enzymes, peptides, and/or polypeptides described herein. Cell lysates may include cells that have been lysed with a lysing agent or lysates such as rabbit reticulocyte or wheat germ lysates. Sample may also include cell-free expression systems. Environmental samples include environmental material such as surface matter, soil, water, crystals, and industrial samples. Such examples are not however to be construed as limiting the sample types applicable to the present invention. Pharmaceutical samples include any therapeutics that are to be tested for the presence and/or concentration of dsRNA, such as RNA-based therapeutics.

As used herein, the terms “fusion,” “fusion polypeptide,” and “fusion protein” refer to a chimeric protein containing a first protein or polypeptide of interest joined to a second different peptide, polypeptide, or protein (e.g., interaction element).

As used herein, the terms “conjugated” and “conjugation” refer to the covalent attachment of two molecular entities (e.g., post-synthesis and/or during synthetic production). The attachment of a peptide or small molecule tag to a protein or small molecule, chemically (e.g., “chemically” conjugated) or enzymatically, is an example of conjugation.

As used herein, the term “modified dehalogenase” refers to a dehalogenase variant (artificial variant) that has one or more mutations that prevent the release of the substrate from the protein following removal of the halogen, resulting in a covalent bond between the substrate and the modified dehalogenase. Because the modified dehalogenase does not release the substrate, it is not capable of turnover, and is not a classical enzyme. The HALOTAG system (Promega) is a commercially available modified dehalogenase and substrate system.

As used herein, the term “bioluminescence” refers to production and emission of light by a chemical reaction catalyzed by, or enabled by, an enzyme, protein, protein complex, or other biomolecule (e.g., bioluminescent complex). In typical embodiments, a substrate for a bioluminescent entity (e.g., bioluminescent protein or bioluminescent complex) is converted into an unstable form by the bioluminescent entity; the substrate subsequently emits light.

As used herein, the term “an Oplophorus luciferase” (“an OgLuc”) refers to a luminescent polypeptide having significant sequence identity, structural conservation, and/or the functional activity of the luciferase produce by and derived from the deep-sea shrimp Oplophorus gracilirostris. In particular, an OgLuc polypeptide refers to a luminescent polypeptide having significant sequence identity, structural conservation, and/or the functional activity of the mature 19 kDa subunit of the Oplophorus luciferase protein complex (e.g., without a signal sequence) such as SEQ ID NOs: 3034 (NanoLuc), which comprises 10 β strands (β1, β2, β3, β4, β5, β6, β7, β8, β9, β10) and utilize substrates such as coelenterazine or a coelenterazine derivative or analog to produce luminescence.

As used herein, the term “B9-like peptide” refers to a peptide (or peptide tag) comprising significant sequence identity, structural conservation, and/or the functional activity of the β (beta) 9 strand of an OgLuc polypeptide. In particular, a β9-like peptide is a peptide capable of structurally complementing an OgLuc polypeptide lacking a β9 strand resulting in enhanced luminescence of the complex compared to the OgLuc polypeptide in the absence of the β9-like peptide. Other “BX-like peptides” may be similarly named (e.g., β1-like, β2-like, β3-like, β4-like, β5-like, β6-like, β7-like, β8-like, β9-like).

As used herein, the term “β10-like peptide” refers to a peptide (or peptide tag) comprising significant sequence identity, structural conservation, and/or the functional activity of the β (beta) 10 strand of an OgLuc polypeptide. In particular, a β10-like peptide is a peptide capable of structurally complementing an OgLuc polypeptide lacking a β10 strand resulting in enhanced luminescence of the complex compared to the OgLuc polypeptide in the absence of the β10-like peptide. Other “BX-like peptides” may be similarly named (e.g., β1-like, β2-like, β3-like, β4-like, β5-like, β6-like, β7-like, β8-like, β9-like).

As used herein, the term “β₁₋₈-like polypeptide” refers to a polypeptide bearing sequence and structural similarity to β (beta) strands 1-8 of an OgLuc polypeptide, but lacking β (beta) strands 9 and 10. Other “β_{Y-Z}-like polypeptides” may be similarly named (e.g., β₁₋₄-like polypeptide, β₂₋₈-like polypeptide, β5-10-like polypeptide, etc.).

As used herein, the term “NANOLUC” refers to an artificial luciferase or bioluminescent polypeptide produced commercially by the Promega Corporation.

As used herein, the term “LgBiT” refers to a polypeptide corresponding to β₁₋₉-like polypeptide that finds use in, for example, binary complementation to form a bioluminescent complex and corresponds to SEQ ID NO: 3037.

As used herein, the term “SmBIT” refers to a peptide corresponding to Bio-like peptide that finds use in, for example, binary complementation to form a bioluminescent complex, but has low affinity for LgBiT (e.g., requires facilitation for complex formation) and corresponds to SEQ ID NO: 3039.

As used herein, the term “HiBiT” refers to a peptide corresponding to Bio-like peptide that finds use in, for example, binary complementation to form a bioluminescent complex, but has high affinity for LgBiT (e.g., does not require facilitation for complex formation). An exemplary HiBiT peptide corresponds to SEQ ID NO: 3038.

As used herein, the term “LgTrip” refers to a polypeptide corresponding to β₁₋₈-like polypeptide. An exemplary LgTrip corresponds to SEQ ID NO: 3045 and finds use in, for example, tripartite complementation with β₉-like and β₁₀-like peptides to form a bioluminescent complex, or binary complementation, with a β₉₋₁₀-like peptide to form a bioluminescent complex.

As used herein, the term “SmTrip10” refers to a peptide corresponding to Bio-like peptide that finds use in, for example, tripartite complementation to form a bioluminescent complex.

As used herein, the term “SmTrip9” refers to a peptide corresponding to β9-like peptide that finds use in, for example, tripartite complementation to form a bioluminescent complex.

As used herein, the term “split” (“sp”) refers to refers to a polypeptide that has been divided into two fragments at an interior site of the original polypeptide. The fragments of a sp polypeptide may reconstitute activity of the original polypeptide if they are structurally complementary and able to form an active complex.

### DETAILED DESCRIPTION

Provided herein are compositions comprising a double-stranded RNA (dsRNA) binding domains linked to components of a complementation system. Upon binding of a pair of dsRNA binding domains to a dsRNA, a detectable complex of the complementation components is formed, and the dsRNA can be detected/quantified.

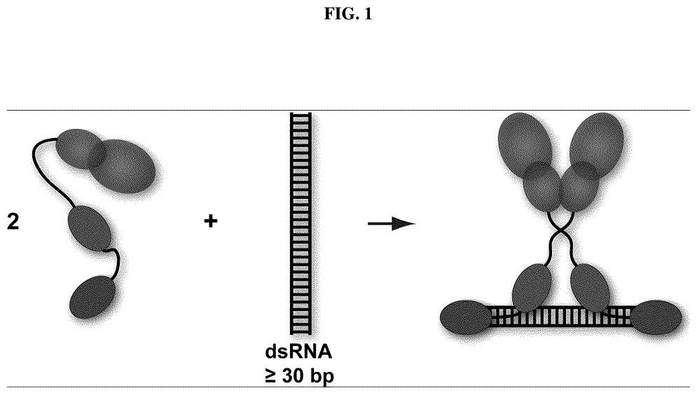

Protein kinase R (PKR, Uniprot #P19525) is an intracellular dsRNA sensor that contains dsRNA binding domains (FIG. 1, blue and red). Upon dimerization of PKR on dsRNA, the kinase domains (FIG. 1, green) are activated, initiating downstream signaling pathways that activate inflammatory pathways and shut down protein translation. PKR binds dsRNA in manner that is independent of target sequence and requires only that the dsRNA be at least 30 base pairs in length (no upper limit). PKR binds the phosphate backbone of dsRNA and does not interact with the nucleotide bases (see, e.g., Nanduri et al., EMBO J, 1998; incorporated by reference in its entirety).

Provided herein are polypeptide constructs that take advantage of the dsRNA binding functionality of PKR and use it to facilitate formation of a detectable complex in the presence of dsRNA. In some embodiments, a pair of components of a detectable complex are fused to PKR dsRNA binding domains (or variants thereof). Upon binding of the dsRNA binding domains to a dsRNA (but not in the absence of a dsRNA), the detectable complex components interact to form the detectable complex and an associated signal is generated. The presence and/or amount of dsRNA in a sample (e.g., environmental, biological, pharmaceutical, etc.) can be detected/quantified based on the signal generated by the systems herein.

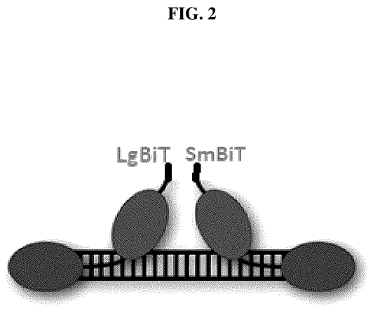

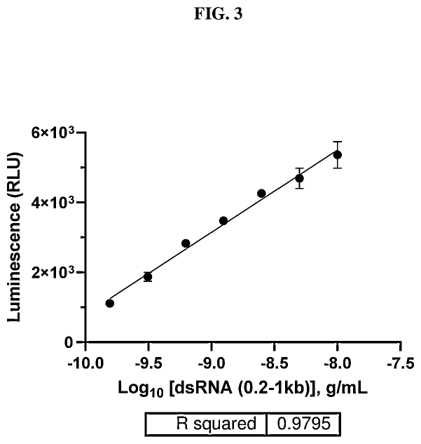

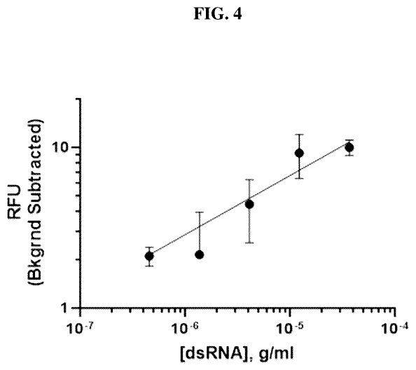

Experiments were conducted during development of embodiments herein to test exemplary systems within the scope herein. First, the commercially available LgBIT and SmBIT components of the NanoBiT system (Promega Corp, Madison, WI) were fused to dsRNA binding domain of PKR (See, e.g., FIG. 2), and luminescence was detected in the presence of furimazine substrate and increasing concentration of a dsRNA (FIG. 3). Second, a split GFP (LgGFP and SmGFP) were fused to dsRNA binding domain of PKR and fluorescence was detected in the presence of increasing concentration of a dsRNA (FIG. 4). These experiments demonstrate that the dsRNA binding domain of PKR can be used to facilitate structural complementation between complementary peptide(s)/polypeptide(s) and form detectable complexes and signal in a dsRNA concentration dependent manner. Provided herein are systems linking any suitable components of detectable complexes to dsRNA binding domains and the use of such systems for the detection of dsRNA in a sample.

I. PKR dsRNA Binding Domains

Interferon (IFN)-induced double-stranded RNA (dsRNA)-activated protein kinase R (PKR) is an IFN-stimulated gene (Gale, M. Jr., and Katze, M. G. (1998). Pharmacol. Ther. 78, 29-46; Peters, G. A., Hartmann, R., Qin, J., and Sen, G. C. (2001). Mol. Cell. Biol. 21, 1908-1920; Pindel, A., and Sadler, A. (2011). J. Interferon Cytokine Res. 31, 59-70; incorporated by reference in their entireties) and acts as a pathogen recognition receptor (Gilfoy, F. D., and Mason, P. W. (2007). J. Virol. 81, 11148-11158; incorporated by reference in its entirety) by recognizing dsRNA, a typical by-product of viral infection, for IFN induction. PKR consists of two functionally distinct domains: an N-terminal regulatory domain and a C-terminal catalytic kinase domain. The regulatory domain contains two dsRNA-binding motifs; binding of dsRNA induces PKR dimerization and allows the exposure of the catalytic site, autophosphorylation, and activation of the kinase (Wu, S., and Kaufman, R. J. (1997). J. Biol. Chem. 272, 1291-1296; Nanduri, et al. (2000). EMBO J. 19, 5567-5574; Dar et al. (2005). Cell 122, 887-900 Dey et al. (2005). Cell 122, 901-913; incorporated by reference in their entireties). Activated PKR catalyzes the phosphorylation of the regulatory α-subunit of the eukaryotic translation initiation factor 2 (cIF2α; Meurs, et al. (1992). J. Virol. 66, 5805-5814; Clemens, M. J., and Elia, A. (1997). J. Interferon Cytokine Res. 17, 503-524; incorporated by reference in their entireties), consequently blocking the initiation of mRNA translation, which results in the global arrest of both cellular and viral protein synthesis and can lead to apoptosis in response to virus infection (Balachandran, et al. (1998). EMBO J. 17, 6888-6902; incorporated by reference in its entirety).

In some embodiments, compositions are provided herein comprising a fusion of a dsRNA binding domain and a component of a detectable complex. In some embodiments, the dsRNA binding domain is capable of binding dsRNA in a sequence agnostic manner. In some embodiments, the dsRNA binding domain does not bind preferentially to the ends of dsRNA or to specific RNA structures. In some embodiments, the dsRNA binding domain binds to dsRNA, but not to single stranded RNA (ssRNA) or DNA (double or single stranded). In some embodiments, the dsRNA binding domain binds to ssRNA and/or DNA at a low enough level that any signal produced from such binding is within the background of an assay herein.

In some embodiments, the dsRNA binding domain of a fusion or system herein corresponds to a PKR dsRNA binding domain. In some embodiments, a dsRNA binding domain of a fusion herein comprises at least 70% (e.g., 70%, 75%, 80%, 85%, 90%, 95%, 100%, or ranges therebetween) sequence identity to SEQ ID NO: 3061 (PKR dsRNA binding domain). In some embodiments, a dsRNA binding domain of a fusion herein comprises at least 70% (e.g., 70%, 75%, 80%, 85%, 90%, 95%, 100%, or ranges therebetween) sequence similarity to SEQ ID NO: 3061 (PKR dsRNA binding domain).

In some embodiments, the dsRNA binding domain of a fusion or system herein comprises a portion or portions that correspond to a portion of the PKR dsRNA binding domain. In some embodiments, the dsRNA binding domain of a fusion or system herein comprises a portion that correspond to the first dsRNA binding motif of the PKR dsRNA binding domain (SEQ ID NO: 3062). In some embodiments, all or a portion of a dsRNA binding domain of a fusion herein comprises at least 70% (e.g., 70%, 75%, 80%, 85%, 90%, 95%, 100%, or ranges therebetween) sequence identity to SEQ ID NO: 3062 (PKR dsRNA binding motif 1). In some embodiments, all or a portion of a dsRNA binding domain of a fusion herein comprises at least 70% (e.g., 70%, 75%, 80%, 85%, 90%, 95%, 100%, or ranges therebetween) sequence similarity to SEQ ID NO: 3062 (PKR dsRNA binding motif 1). In some embodiments, the dsRNA binding domain of a fusion or system herein comprises a portion that correspond to the second dsRNA binding motif of the PKR dsRNA binding domain (SEQ ID NO: 3063). In some embodiments, all or a portion of a dsRNA binding domain of a fusion herein comprises at least 70% (e.g., 70%, 75%, 80%, 85%, 90%, 95%, 100%, or ranges therebetween) sequence identity to SEQ ID NO: 3063 (PKR dsRNA binding motif 2). In some embodiments, all or a portion of a dsRNA binding domain of a fusion herein comprises at least 70% (e.g., 70%, 75%, 80%, 85%, 90%, 95%, 100%, or ranges therebetween) sequence similarity to SEQ ID NO: 3063 (PKR dsRNA binding motif 2). In some embodiments, a dsRNA binding domain of a fusion herein comprises a first segment with at least 70% (e.g., 70%, 75%, 80%, 85%, 90%, 95%, 100%, or ranges therebetween) sequence identity to SEQ ID NO: 3062 (PKR dsRNA binding motif 1) and a second segment with at least 70% (e.g., 70%, 75%, 80%, 85%, 90%, 95%, 100%, or ranges therebetween) sequence identity to SEQ ID NO: 3063 (PKR dsRNA binding motif 2). In some embodiments, a dsRNA binding domain of a fusion herein comprises a first segment with at least 70% (e.g., 70%, 75%, 80%, 85%, 90%, 95%, 100%, or ranges therebetween) sequence similarity to SEQ ID NO: 3062 (PKR dsRNA binding motif 1) and a second segment with at least 70% (e.g., 70%, 75%, 80%, 85%, 90%, 95%, 100%, or ranges therebetween) sequence similarity to SEQ ID NO: 3063 (PKR dsRNA binding motif 2). In some embodiments, segments corresponding to PKR dsRNA binding motif 1 and PKR dsRNA binding motif 2 (or variants thereof) are fused by a linker of 1-100 amino acids in length. In some embodiments, the linker comprises the natural linker present in the PKR dsRNA binding domain.

### II. Detectable Complexes

In some embodiments, the systems herein comprise a pair of fusions: (1) a first fusion comprising a dsRNA binding domain linked to a first component of a detectable complex, and (2) a second fusion comprising a dsRNA binding domain linked to a second component of a detectable complex. In some embodiments, the components of the complex have a sufficiently low enough affinity for one another that a facilitation is required in order to form a complex and produce a detectable signal (or the signal produced upon facilitation is significantly greater than the unfacilitated signal such that the unfacilitated signal resides in the background). In some embodiments, the binding of the dsRNA binding domains linked to the components of the detectable complex provides facilitation to form the detectable complex and produce the detectable signal.

In some embodiments, the complex may produce any suitable signal that allows for detection of binding of a pair of complementary fusions herein to dsRNA. For example, upon complementation, a detectable complex may produce any suitable signal, such as fluorescence, luminescence, enzymatic activity, ligand binding, etc. In some embodiments, the components correspond to fragments of an enzyme or other protein capable of producing a detectable signal (or variants of such fragments). In such embodiments, upon facilitation by binding of the dsRNA binding domains to a dsRNA, the components form a complex that is analogous to the enzyme or other protein, and a corresponding signal can be detected. In other embodiments, the components may form a detectable complex that does not correspond to an existing enzyme or complex.

In some embodiments, the detectable complex corresponds to a protein that has been split into two fragments that are capable of non-covalently interacting to form a complex that exhibits the functional activity of the protein (See, e.g., Shekhawat & Ghosh. Curr Opin Chem Biol. 2011 December; 15 (6): 789-797; incorporated by reference in its entirety). In some embodiments, a suitable protein is one with a detectable activity that that is reversibly eliminated by fragmenting the protein into two separate components, wherein the activity is reconstituted when the fragments are non-covalently reassociated through the binding of dsRNA binding domains fused to the respective fragments to a dsRNA. Any protein that can be split into fragments that can associate (e.g., with facilitation) to reconstitute the activity of the parent protein could find use in embodiments herein. Examples of such proteins include beta-lactamase (Galarneau et al. Nat. Biotech. 2002; 20 (6): 619-622; incorporated by reference in its entirety), ubiquitin (Johnsson & Varshavsky A. Proc Natl Acad Sci USA. 1994; 91:10340-13044; incorporated by reference in its entirety), dihydrofolate reductase (DHFR) (Pelletier et al. Proc Natl Acad Sci USA. 1998; 95:12141-12146; incorporated by reference in its entirety), focal adhesion kinase (FAK), Gal4, GFP and variants (Ghosh et al. J. Am. Chem. Soc. 2000; 122:5658-5659; Hu & Kerppola. Nat. Biotechnol. 2003; 21:539-1545; incorporated by reference in their entireties) (e.g., EGFP), horseradish peroxidase, infrared fluorescent protein, various luciferases (Remy & Michnick. Nat. Meth. 2006; 3 (12): 977-979; Paulmurugan & Gambhir. Ana. Chem. 2003; 759 (7): 1584-1589; incorporated by reference in their entireties) (e.g., recombinase enhanced bimolecular luciferase, Gaussia princeps luciferase, Firefly (Luker et al. Proc Natl Acad Sci USA. 2004; 101:12288-122893; incorporated by reference in its entirety), etc.), tobacco etch virus (TEV) protease (Wehr et al. Nat. Meth. 2006; 3 (12): 985-993; incorporated by reference in its entirety), thymidine kinase (Massoud et al. Nat. Med. 2010; 16 (8): 921-927; incorporated by reference in its entirety), chorismate mutase (Muller et al. Prot. Sci. 2010; 19 (5): 1000-1010; incorporated by reference in its entirety), etc. In some embodiments, the components of a complex used in embodiments herein are variants of the fragments of a protein (e.g., less than 100% sequence identity) but are capable of producing activity similar to that of the protein upon complex formation. Other examples of detectable complex components are described below.

### A. HaloTag

In some embodiments, provided herein are fusions of dsRNA bounding domains with complementary pairs of peptides/polypeptides capable of interacting with each other (e.g., facilitated by binding of dsRNA binding domains to a dsRNA) to form an active modified dehalogenase complex capable of forming a covalent bond to a haloalkane ligand. In some embodiments, a first fusion is provided comprising a first complementary peptide or polypeptide fragment of a modified dehalogenase, and a second fusion is provided comprising a second complementary peptide or polypeptide fragment of the modified dehalogenase, wherein upon interacting (e.g., facilitated by binding of the dsRNA bounding domains to a dsRNA), the complementary peptide(s)/polypeptide(s) form an active modified dehalogenase complex capable of forming a covalent bond to a haloalkane ligand. In some embodiments, the complementary peptide(s)/polypeptide(s) are fragments of a split mutant dehalogenase.

In some embodiments, the peptide/polypeptide components capable of forming a modified dehalogenase complex are fragments of a split mutated dehalogenase, such as those derived from the commercially available HALOTAG protein (Promega) and/or mutated dehalogenases disclosed in U.S. published application 2006/0024808, the disclosure of which is incorporated by reference herein.

In some embodiments, provided as a component of the compositions, systems, and methods herein are split modified dehalogenases. In some embodiments, a first fragment of a mutant dehalogenase is fused to a first dsRNA binding domain, and a second fragment of the mutant dehalogenase is fused to a second dsRNA binding domain. In some embodiments, at least one of the mutant dehalogenase fragments has a substitution that if present in a full-length modified dehalogenase (or a corresponding complex), forms a covalent bond with a haloalkane ligand. In some embodiments, the first fragment of the mutant dehalogenase and the second fragment of the mutant dehalogenase are capable of interacting (e.g., facilitated by binding of linked dsRNA binding domains to a dsRNA) to form an active modified dehalogenase complex.

HALOTAG is a 297-residue self-labeling polypeptide (33 kDa) derived from a bacterial hydrolase (dehalogenase) enzyme, which has been modified to covalently bind to its ligand, a haloalkane moiety. The HALOTAG ligand can be linked to solid surfaces (e.g., beads) or functional groups (e.g., fluorophores), and the HALOTAG polypeptide can be fused to various proteins of interest, allowing covalent attachment of the protein of interest to the solid surface or functional group. The HALOTAG polypeptide is a modified dehalogenase with a genetically modified active site, which specifically binds to the haloalkane ligand chloroalkane linker with an enhanced and increased rate of ligand binding (Pries et al. The Journal of Biological Chemistry. 270 (18): 10405-11; incorporated by reference in its entirety). The reaction that forms the bond between the protein tag and chloroalkane linker is fast and essentially irreversible under physiological conditions (Waugh DS (June 2005). Trends in Biotechnology. 23 (6): 316-20; incorporated by reference in its entirety). In the natural hydrolase enzyme, nucleophilic attack of the chloroalkane reactive linker causes displacement of the halogen with an amino acid residue, which results in the formation of a covalent alkyl-enzyme intermediate. This intermediate would then be hydrolyzed by an amino acid residue within the wild-type hydrolase (Chen et al. (February 2005) Current Opinion in Biotechnology. 16 (1): 35-40; incorporated by reference in its entirety). This would lead to regeneration of the enzyme following the reaction. However, with HALOTAG, the modified haloalkane dehalogenase, the reaction intermediate cannot proceed through the second reaction because it cannot be hydrolyzed due to the mutation in the enzyme. This causes the intermediate to persist as a stable covalent adduct with which there is no associated back reaction (Marks et al. (August 2006) Nature Methods. 3 (8): 591-6; incorporated by reference in its entirety). Various HALOTAG ligands, functional groups, fusions, assays, modifications, uses, etc. are described in U.S. Pat. Nos. 8,748,148; 9,593,316; 10,246,690; 8,742,086; 9,873,866; 10,604,745; U.S. Pat. App. 2009/0253131; U.S. Pat. App. 2010/0273186; 20130337539; U.S. Pat. App. 2012/0258470; U.S. Pat. App. 2012/0252048; U.S. Pat. App. 2011/0201024; U.S. 2014/0322794; each of which is incorporated by reference in their entireties.

In some embodiments, the fragments, complementary peptides, complementary polypeptides, etc., of a modified dehalogenase described herein are a HALOTAG-based complementation system. In some embodiments, the fragments, complementary peptides, complementary polypeptides, etc., of a modified dehalogenase described herein correspond (e.g., sequence identity, sequence similarity, 3D structure, etc.) to sequences within the HALOTAG protein. In some embodiments, a modified dehalogenase complex herein, comprising two or more peptide or polypeptide components corresponds to a HALOTAG protein and is capable of binding to a halkoalkyl ligand in a similar manner.

In some embodiments, as described in U.S. Prov. App. No. 63/338,323 and PCT App. No. PCT/US23/20959, both of which are herein incorporated by reference in their entireties, extensive experiments have been conducted to demonstrate the feasibility of generating fragments of HALOTAG (and variants thereof) capable of interacting to form a modified dehalogenase complex capable of binding to a haloalkyl ligand, as well as optimizing variants of HALOTAG fragments for desired characteristics. As described herein, embodiments are not limited to the HALOTAG sequences. In some embodiments, provided herein are split modified dehalogenases (e.g., as fusions with dsRNA binding domains) that differ in sequence from HALOTAG (SEQ ID NO: 1).

In some embodiments, compositions and systems are provided comprising components of a split modified dehalogenase, such as a split HALOTAG (“spHT”) or variants thereof (e.g., as fusions with dsRNA binding domains). In some embodiments, systems and compositions herein comprise spHT peptides and polypeptides (e.g., as a portion of the fusions described herein).

In some embodiments, compositions (e.g., fusions) and systems (e.g., multiple fusions with appropriate ligands and substrates) are provided comprising polypeptides, peptides, fragments, and combinations thereof derived from a modified dehalogenase sequence of SEQ ID NO: 1 (HALOTAG):

MAEIGTGFPFDPHYVEVLGERMHYVDVGPRDGTPVLFLHGNPTSSYVWRN
IIPHVAPTHRCIAPDLIGMGKSDKPDLGYFFDDHVRFMDAFIEALGLEEV
VLVIHDWGSALGFHWAKRNPERVKGIAFMEFIRPIPTWDEWPEFARETFQ
AFRTTDVGRKLIIDQNVFIEGTLPMGVVRPLTEVEMDHYREPFLNPVDRE
PLWRFPNELPIAGEPANIVALVEEYMDWLHQSPVPKLLFWGTPGVLIPPA
EAARLAKSLPNCKAVDIGPGLNLLQEDNPDLIGSEIARWLSTLEISG.

In some embodiments, spHT peptides and polypeptides herein (e.g., as portions of fusions with dsRNA binding domains) comprise at least 70% sequence identity with a portion of SEQ ID NO: 1 (e.g., >70% sequence identity, >75% sequence identity, >80% sequence identity, >85% sequence identity, >90% sequence identity, >95% sequence identity, >96% sequence identity, >97% sequence identity, >98% sequence identity, >99% sequence identity). In some embodiments, spHT peptides and polypeptides (e.g., as portions of fusions with dsRNA binding domains) comprise 100% sequence identity with all or a portion of SEQ ID NO: 1. In some embodiments, spHT peptides and polypeptides herein (e.g., as portions of fusions with dsRNA binding domains) comprise at least 70% sequence similarity with all or a portion of SEQ ID NO: 1 (e.g., >70% sequence similarity, >75% sequence similarity, >80% sequence similarity, >85% sequence similarity, >90% sequence similarity, >95% sequence similarity, >96% sequence similarity, >97% sequence similarity, >98% sequence similarity, >99% sequence similarity). In some embodiments, spHT peptides and polypeptides herein (e.g., as portions of fusions with dsRNA binding domains) comprise 100% sequence similarity with all or a portion of SEQ ID NO: 1.

In some embodiments, spHT peptides or polypeptides (e.g., as portions of fusions with dsRNA binding domains) herein comprise an A at a position corresponding to position 2 of SEQ ID NO: 1. In other embodiments, spHT peptides or polypeptides (e.g., as portions of fusions with dsRNA binding domains) herein comprise an S at a position corresponding to position 2 of SEQ ID NO: 1. In some embodiments, spHT peptides or polypeptides (e.g., as portions of fusions with dsRNA binding domains) herein comprise a V at a position corresponding to position 47 of SEQ ID NO: 1. In some embodiments, spHT peptides or polypeptides (e.g., as portions of fusions with dsRNA binding domains) herein comprise a T at a position corresponding to position 58 of SEQ ID NO: 1. In some embodiments, spHT peptides or polypeptides (e.g., as portions of fusions with dsRNA binding domains) herein comprise a G at a position corresponding to position 78 of SEQ ID NO: 1. In some embodiments, spHT peptides or polypeptides (e.g., as portions of fusions with dsRNA binding domains) herein comprise a F at a position corresponding to position 88 of SEQ ID NO: 1. In some embodiments, spHT peptides or polypeptides (e.g., as portions of fusions with dsRNA binding domains) herein comprise a M at a position corresponding to position 89 of SEQ ID NO: 1. In some embodiments, spHT peptides or polypeptides (e.g., as portions of fusions with dsRNA binding domains) herein comprise a F at a position corresponding to position 128 of SEQ ID NO: 1. In some embodiments, spHT peptides or polypeptides (e.g., as portions of fusions with dsRNA binding domains) herein comprise a T at a position corresponding to position 155 of SEQ ID NO: 1. In some embodiments, spHT peptides or polypeptides (e.g., as portions of fusions with dsRNA binding domains) herein comprise a K at a position corresponding to position 160 of SEQ ID NO: 1. In some embodiments, spHT peptides or polypeptides (e.g., as portions of fusions with dsRNA binding domains) herein comprise a V at a position corresponding to position 167 of SEQ ID NO: 1. In some embodiments, spHT peptides or polypeptides (e.g., as portions of fusions with dsRNA binding domains) herein comprise a T at a position corresponding to position 172 of SEQ ID NO: 1. In some embodiments, spHT peptides or polypeptides (e.g., as portions of fusions with dsRNA binding domains) herein comprise a M at a position corresponding to position 175 of SEQ ID NO: 1. In some embodiments, spHT peptides or polypeptides (e.g., as portions of fusions with dsRNA binding domains) herein comprise a G at a position corresponding to position 176 of SEQ ID NO: 1. In some embodiments, spHT peptides or polypeptides (e.g., as portions of fusions with dsRNA binding domains) herein comprise a N at a position corresponding to position 195 of SEQ ID NO: 1. In some embodiments, spHT peptides or polypeptides (e.g., as portions of fusions with dsRNA binding domains) herein comprise a E at a position corresponding to position 224 of SEQ ID NO: 1. In some embodiments, spHT peptides or polypeptides (e.g., as portions of fusions with dsRNA binding domains) herein comprise a D at a position corresponding to position 227 of SEQ ID NO: 1. In some embodiments, spHT peptides or polypeptides (e.g., as portions of fusions with dsRNA binding domains) herein comprise a K at a position corresponding to position 257 of SEQ ID NO: 1. In some embodiments, spHT peptides or polypeptides (e.g., as portions of fusions with dsRNA binding domains) herein comprise an A at a position corresponding to position 264 of SEQ ID NO: 1. In some embodiments, peptides or polypeptides herein comprise a N at a position corresponding to position 272 of SEQ ID NO: 1. In some embodiments, spHT peptides or polypeptides (e.g., as portions of fusions with dsRNA binding domains) herein comprise a L at a position corresponding to position 273 of SEQ ID NO: 1. In some embodiments, spHT peptides or polypeptides (e.g., as portions of fusions with dsRNA binding domains) herein comprise a S at a position corresponding to position 291 of SEQ ID NO: 1. In some embodiments, spHT peptides or polypeptides (e.g., as portions of fusions with dsRNA binding domains) herein comprise a T at a position corresponding to position 292 of SEQ ID NO: 1. In some embodiments, spHT peptides or polypeptides (e.g., as portions of fusions with dsRNA binding domains) herein comprise a E at a position corresponding to position 294 of SEQ ID NO: 1. In some embodiments, spHT peptides or polypeptides (e.g., as portions of fusions with dsRNA binding domains) herein comprise a I at a position corresponding to position 295 of SEQ ID NO: 1. In some embodiments, spHT peptides or polypeptides (e.g., as portions of fusions with dsRNA binding domains) herein comprise a S at a position corresponding to position 296 of SEQ ID NO: 1. In some embodiments, spHT peptides or polypeptides (e.g., as portions of fusions with dsRNA binding domains) herein comprise a G at a position corresponding to position 297 of SEQ ID NO: 1.

In some embodiments, spHT peptides or polypeptides (e.g., as portions of fusions with dsRNA binding domains) herein do not have an S at a position corresponding to position 2 of SEQ ID NO: 1. In some embodiments, spHT peptides or polypeptides (e.g., as portions of fusions with dsRNA binding domains) herein do not have a L at a position corresponding to position 47 of SEQ ID NO: 1. In some embodiments, spHT peptides or polypeptides (e.g., as portions of fusions with dsRNA binding domains) herein do not have a S at a position corresponding to position 58 of SEQ ID NO: 1. In some embodiments, spHT peptides or polypeptides (e.g., as portions of fusions with dsRNA binding domains) herein do not have a D at a position corresponding to position 78 of SEQ ID NO: 1. In some embodiments, spHT peptides or polypeptides (e.g., as portions of fusions with dsRNA binding domains) herein do not have a Y at a position corresponding to position 88 of SEQ ID NO: 1. In some embodiments, spHT peptides or polypeptides (e.g., as portions of fusions with dsRNA binding domains) herein do not have a L at a position corresponding to position 89 of SEQ ID NO: 1. In some embodiments, spHT peptides or polypeptides (e.g., as portions of fusions with dsRNA binding domains) herein do not have a C at a position corresponding to position 128 of SEQ ID NO: 1. In some embodiments, spHT peptides or polypeptides (e.g., as portions of fusions with dsRNA binding domains) herein do not have an A at a position corresponding to position 155 of SEQ ID NO: 1. In some embodiments, spHT peptides or polypeptides (e.g., as portions of fusions with dsRNA binding domains) herein do not have a E at a position corresponding to position 160 of SEQ ID NO: 1. In some embodiments, spHT peptides or polypeptides (e.g., as portions of fusions with dsRNA binding domains) herein do not have an A at a position corresponding to position 167 of SEQ ID NO: 1. In some embodiments, spHT peptides or polypeptides (e.g., as portions of fusions with dsRNA binding domains) herein do not have an A at a position corresponding to position 172 of SEQ ID NO: 1. In some embodiments, spHT peptides or polypeptides (e.g., as portions of fusions with dsRNA binding domains) herein do not have a K at a position corresponding to position 175 of SEQ ID NO: 1. In some embodiments, spHT peptides or polypeptides (e.g., as portions of fusions with dsRNA binding domains) herein do not have a C at a position corresponding to position 176 of SEQ ID NO: 1. In some embodiments, spHT peptides or polypeptides (e.g., as portions of fusions with dsRNA binding domains) herein do not have a K at a position corresponding to position 195 of SEQ ID NO: 1. In some embodiments, spHT peptides or polypeptides (e.g., as portions of fusions with dsRNA binding domains) herein do not have an A at a position corresponding to position 224 of SEQ ID NO: 1. In some embodiments, spHT peptides or polypeptides (e.g., as portions of fusions with dsRNA binding domains) herein do not have a N at a position corresponding to position 227 of SEQ ID NO: 1. In some embodiments, spHT peptides or polypeptides (e.g., as portions of fusions with dsRNA binding domains) herein do not have a E at a position corresponding to position 257 of SEQ ID NO: 1. In some embodiments, spHT peptides or polypeptides (e.g., as portions of fusions with dsRNA binding domains) herein do not have a T at a position corresponding to position 264 of SEQ ID NO: 1. In some embodiments, spHT peptides or polypeptides (e.g., as portions of fusions with dsRNA binding domains) herein do not have a H at a position corresponding to position 272 of SEQ ID NO: 1. In some embodiments, spHT peptides or polypeptides (e.g., as portions of fusions with dsRNA binding domains) herein do not have a Y at a position corresponding to position 273 of SEQ ID NO: 1. In some embodiments, spHT peptides or polypeptides (e.g., as portions of fusions with dsRNA binding domains) herein do not have a P at a position corresponding to position 291 of SEQ ID NO: 1. In some embodiments, spHT peptides or polypeptides (e.g., as portions of fusions with dsRNA binding domains) herein do not have an A at a position corresponding to position 292 of SEQ ID NO: 1. In some embodiments, spHT peptides or polypeptides (e.g., as portions of fusions with dsRNA binding domains) herein do not have an amino acid at a position corresponding to position 294 of SEQ ID NO: 1. In some embodiments, spHT peptides or polypeptides (e.g., as portions of fusions with dsRNA binding domains) herein do not have an amino acid at a position corresponding to position 295 of SEQ ID NO: 1. In some embodiments, spHT peptides or polypeptides (e.g., as portions of fusions with dsRNA binding domains) herein do not have an amino acid at a position corresponding to position 296 of SEQ ID NO: 1. In some embodiments, spHT peptides or polypeptides (e.g., as portions of fusions with dsRNA binding domains) herein do not have an amino acid at a position corresponding to position 297 of SEQ ID NO: 1.

In some embodiments, a sp dehalogenase comprises two peptide and/or polypeptide components that collectively comprise at least 70% sequence similarity or identity with all or a portion of SEQ ID NO: 1 (e.g., >70% sequence similarity or identity, >75% sequence similarity or identity, >80% sequence similarity or identity, >85% sequence similarity or identity, >90% sequence similarity or identity, >95% sequence similarity or identity, >96% sequence similarity or identity, >97% sequence similarity or identity, >98% sequence similarity or identity, >99% sequence similarity or identity). For example, the first peptide/polypeptide component of the sp polypeptide corresponds to a first portion of SEQ ID NO: 1 (e.g., at least 70% sequence similarity or identity to the first portion), and the second peptide/polypeptide component of the sp polypeptide corresponds to a second portion of SEQ ID NO: 1 (e.g., at least 70% sequence similarity or identity to the second portion). In some embodiments, a sp dehalogenase (e.g., spHT) comprises two fragments that collectively comprise 100% sequence similarity or identity with all or a portion of SEQ ID NO: 1. For example, the first fragment of the sp polypeptide has 100% sequence similarity or identity to a first portion of SEQ ID NO: 1, and the second fragment of the sp polypeptide has 100% sequence similarity or identity to a second portion SEQ ID NO: 1.

In some embodiments, a sp dehalogenase (e.g., as portions of fusions with dsRNA binding domains) comprises a sp site. The sp site is an internal location in the parent sequence that defines the C-terminus of the first component or fragment and the N-terminus of the second component or fragment of the sp dehalogenase. For example, if a theoretical a 100 amino acid polypeptide were split with a sp site between residues 57 and 58 of the parent polypeptide (referred to herein as a sp site of 57), the first component polypeptide would correspond to positions 1-57, and the second component polypeptide would correspond to positions 58-100. In some embodiments herein, a sp site within SEQ ID NO: 1 may occur at any position from position 5 of SEQ ID NO: 1 to position 290 of SEQ ID NO: 1. In some embodiments, SEQ ID NOS: 2-577 are exemplary components of spHT polypeptides having 100% sequence identity to SEQ ID NO: 1. In some embodiments, an active spHT complex is formed between two fragments that collectively comprise amino acids corresponding to each position in SEQ ID NO: 1. For example, a polypeptide having a sequence of SEQ ID NO: 26 and a peptide having a sequence of SEQ ID NO: 27 collectively comprise amino acids corresponding to each position in SEQ ID NO: 1. Any pairs of peptide and polypeptides (or two polypeptides) corresponding to two of SEQ ID NOS: 2-577 and together comprising amino acids corresponding to each position in SEQ ID NO: 1 (with or without deletion or duplication of positions) find use in embodiments herein. In some embodiments, a spHT dehalogenase comprises any of the following pairs of fragments (e.g., fused to dsRNA binding domains): SEQ ID NOS: 2 and 3, 4 and 5, 6 and 7, 8 and 9, 10 and 11, 12 and 13, 14 and 15, 16 and 17, 18 and 19, 20 and 21, 22 and 23, 24 and 25, 26 and 27, 28 and 29, 30 and 31, 32 and 33, 34 and 35, 36 and 37, 38 and 39, 40 and 41, 42 and 43, 44 and 45, 46 and 47, 48 and 49, 50 and 51, 52 and 53, 54 and 55, 56 and 57, 58 and 59, 60 and 61, 62 and 63, 64 and 65, 66 and 67, 68 and 69, 70 and 71, 72 and 73, 74 and 75, 76 and 77, 78 and 79, 80 and 81, 82 and 83, 84 and 85, 86 and 87, 88 and 89, 90 and 91, 92 and 93, 94 and 95, 96 and 97, 98 and 99, 100 and 101, 102 and 103, 104 and 105, 106 and 107, 108 and 109, 110 and 111, 112 and 113, 114 and 115, 116 and 117, 118 and 119, 120 and 121, 121, 122 and 123, 124 and 125, 126 and 127, 128 and 129, 130 and 131, 132 and 133, 134 and 135, 136 and 137, 138 and 139, 140 and 141, 142 and 143, 144 and 145, 146 and 147, 148 and 149, 150 and 151, 152 and 153, 154 and 155, 156 and 157, 158 and 159, 160 and 161, 172 and 173, 174 and 175, 176 and 177, 178 and 179, 180 and 181, 182 and 183, 184 and 185, 186 and 187, 188 and 189, 190 and 191, 192 and 193, 194 and 195, 196 and 197, 198 and 199, 200 and 201, 202 and 203, 204 and 205, 206 and 207, 208 and 209, 190 and 211, 212 and 213, 214 and 215, 216 and 217, 218 and 219, 220 and 221, 222 and 223, 224 and 225, 226 and 227, 228 and 229, 300 and 301, 302 and 303, 304 and 305, 306 and 307, 308 and 309, 310 and 311, 312 and 313, 314 and 315, 316 and 317, 318 and 319, 320 and 321, 322 and 323, 324 and 325, 326 and 327, 328 and 329, 330 and 331, 332 and 333, 334 and 335, 336 and 337, 338 and 339, 340 and 341, 342 and 343, 344 and 345, 346 and 347, 348 and 349, 350 and 351, 352 and 353, 354 and 355, 356 and 357, 358 and 359, 360 and 361, 362 and 363, 364 and 365, 366 and 367, 368 and 369, 370 and 371, 372 and 373, 374 and 375, 376 and 377, 378 and 379, 380 and 381, 382 and 383, 384 and 385, 386 and 387, 388 and 389, 390 and 391, 392 and 393, 394 and 395, 396 and 397, 398 and 399, 400 and 401, 402 and 403, 404 and 405, 406 and 407, 408 and 409, 410 and 411, 412 and 413, 414 and 415, 416 and 417, 418 and 419, 420 and 421, 422 and 423, 424 and 425, 426 and 427, 428 and 429, 430 and 431, 432 and 433, 434 and 435, 436 and 437, 438 and 439, 440 and 441, 442 and 443, 444 and 445, 446 and 447, 448 and 449, 450 and 451, 452 and 453, 454 and 455, 456 and 457, 458 and 459, 460 and 461, 462 and 463, 464 and 465, 466 and 467, 468 and 469, 470 and 471, 472 and 473, 474 and 475, 476 and 477, 478 and 479, 480 and 481, 482 and 483, 484 and 485, 486 and 487, 488 and 489, 490 and 491, 492 and 493, 494 and 495, 496 and 497, 498 and 499, 500 and 501, 502 and 503, 504 and 505, 506 and 507, 508 and 509, 510 and 511, 512 and 513, 514 and 515, 516 and 517, 518 and 519, 520 and 521, 522 and 523, 524 and 525, 526 and 527, 528 and 529, 530 and 531, 532 and 533, 534 and 535, 536 and 537, 538 and 539, 540 and 541, 542 and 543, 544 and 545, 546 and 547, 548 and 549, 550 and 551, 552 and 553, 554 and 555, 556 and 557, 558 and 559, 560 and 561, 562 and 563, 564 and 565, 566 and 567, 568 and 569, 570 and 571, 572 and 573, 574 and 575, and 576 and 577.

In some embodiments, a spHT comprises a peptide and polypeptide (or two polypeptides) pair corresponding to two of SEQ ID NOS: 2-577 together comprising amino acids corresponding to each position in SEQ ID NO: 1, but with a deletion of up to 40 amino acids in length (e.g., 1, 2, 3, 4, 5, 6, 7, 8, 9, 10, 15, 20, 25, 30, 35, 40, or ranges therebetween) at the C-terminus or N-terminus of one or both of fragments. For example, a pair corresponding to SEQ ID NOS: 7 and 28 together correspond to positions of SEQ ID NO: 1, but with an 11-residue deletion. In some embodiments, any pairs of SEQ ID NOS: 2-577, together corresponding to the sequence of SEQ ID NO: 1, but with deletions of up to 40 amino acids, are within the scope of spHTs herein. In some embodiments, the deletion is adjacent to the split site. In some embodiments, the deletion corresponds to the N- or C-terminus of SEQ ID NO: 1.

In some embodiments, a spHT comprises a peptide and polypeptide (or two polypeptides) pair corresponding to two of SEQ ID NOS: 2-577 together comprising amino acids corresponding to each position in SEQ ID NO: 1, but with a duplication of up to 40 amino acids in length (e.g., 1, 2, 3, 4, 5, 6, 7, 8, 9, 10, 15, 20, 25, 30, 35, 40, or ranges therebetween) at the C-terminus or N-terminus of one or both of fragments. For example, a pair corresponding to SEQ ID NOS: 6 and 29 together correspond to positions of SEQ ID NO: 1, but with an 11 residue duplication. In some embodiments, any pairs of SEQ ID NOS: 2-577, together corresponding to the sequence of SEQ ID NO: 1, but with duplications of up to 40 amino acids, are within the scope of spHTs herein. In some embodiments, the duplication is adjacent to the split site. In some embodiments, the duplication corresponds to the N- or C-terminus of SEQ ID NO: 1.

Fragments utilizing any sp sites, for example, corresponding to a position between position 5 and position 290 of SEQ ID NO: 1, are readily envisioned and within the scope herein.

In some embodiments, spHTs are provided with a sp site corresponding to position 5, 6, 7, 8, 9, 10, 11, 12, 13, 14, 15, 16, 17, 18, 19, 31, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 313, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 310, 311, 312, 313, 314, 315, 316, 317, 318, 319, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, or 290 of SEQ ID NO: 1.

In some embodiments, spHTs are provided with a sp site corresponding to a position between positions 5 and 13, 36 and 51, 63 and 72, 84 and 92, 104 and 130, 142 and 148, 160 and 174, 186 and 189, 311 and 313, 221 and 229, or 269 and 290 of SEQ ID NO: 1.

In some embodiments, the spHT peptides and polypeptides herein (e.g., fused to dsRNA binding domains) comprise one or more (e.g., 2, 3, 4, 5, 6, 7, 8, 9 10, 11, 12, 13, 14, 15, 16, 17, 18, 19m 20, 25, 30, 35, 40, 50, 75, or more) substitutions or deletions relative to one of SEQ ID NOS: 2-557. In some embodiments, sp peptides and polypeptides are provided (e.g., fused to dsRNA binding domains) having 70%-100% sequence identity to one of SEQ ID NOS: 2-557 (e.g., >70% sequence identity, >75% sequence identity, >80% sequence identity, >85% sequence identity, >90% sequence identity, >95% sequence identity, >96% sequence identity, >97% sequence identity, >98% sequence identity, >99% sequence identity). In some embodiments, sp peptides and polypeptides are provided (e.g., fused to dsRNA binding domains) having 70%-100% sequence similarity to one of SEQ ID NOS: 2-557 (e.g., >70% sequence similarity, >75% sequence similarity, >80% sequence similarity, >85% sequence similarity, >90% sequence similarity, >95% sequence similarity, >96% sequence similarity, >97% sequence similarity, >98% sequence similarity, >99% sequence similarity).

In some embodiments, pairs of sp peptides and/or polypeptides are provided (e.g., fused to dsRNA binding domains) that are capable of forming active sp dehalogenase complexes (active spHT complexes). In some embodiments, such pairs comprise at least 70% sequence identity or similarity to two of SEQ ID NOS: 2-557, and together comprise residues corresponding to 100% of the positions in SEQ ID NO: 1, allowing for up to 40 deletions or duplications at the C- or N-terminus of the peptides/polypeptides.

In some embodiments, the first fragment of a spHT complementary pair (e.g., fused to a dsRNA binding domain) corresponds to position 1 through position 5, 6, 7, 8, 9, 10, 11, 12, 13, 14, 15, 16, 17, 18, 19, 31, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 313, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 310, 311, 312, 313, 314, 315, 316, 317, 318, 319, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, or 290 of SEQ ID NO: 1.

In some embodiments, the second fragment of a spHT complementary pair (e.g., fused to a dsRNA binding domain) corresponds to position 7, 8, 9, 10, 11, 12, 13, 14, 15, 16, 17, 18, 19, 31, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 313, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 310, 311, 312, 313, 314, 315, 316, 317, 318, 319, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, or 290 through position 294 of SEQ ID NO: 1.

In some embodiments, the duplicated portion of a spHT complementary pair is 1-40 amino acids in length (e.g., 1, 2, 3, 4, 5, 6, 7, 8, 9, 10, 11, 12, 13, 14, 15, 16, 17, 18, 19, 31, 21, 22, 23, 24, 25, 26, 27, 28, 29, 30, 31, 32, 33, 34, 35, 36, 37, 38, 39, 40, or ranges therebetween).

In some embodiments, the deleted portion of a spHTs complementary pair is 1-40 amino acids in length (e.g., 1, 2, 3, 4, 5, 6, 7, 8, 9, 10, 11, 12, 13, 14, 15, 16, 17, 18, 19, 31, 21, 22, 23, 24, 25, 26, 27, 28, 29, 30, 31, 32, 33, 34, 35, 36, 37, 38, 39, 40, or ranges therebetween).

The exemplary spHT fragment sequences of SEQ ID NOS: 2-577 comprise 100% sequence identity to portions of SEQ ID NO: 1; there are no portions of these sequences that do not align with 100% sequence identity to SEQ ID NO: 1. However, as described herein, spHT peptides and polypeptides may have less than 100% sequence identity with SEQ ID NO: 1 (e.g., >70%, >75%, >80%, >85%, >90%, >95%, >96%, >97%, >98%, >99%, but less than 100% sequence identity). Therefore, peptides and polypeptide having less than 100% sequence identity with one or SEQ ID NOS: 2-577 (e.g., >70%, >75%, >80%, >85%, >90%, >95%, >96%, >97%, >98%, >99%, but less than 100% sequence identity) are provided herein and find use in the complementary pairs and complexes herein.

In some embodiments, a spHT complementary pair herein comprises a peptide corresponding to SEQ ID NO: 578 and a polypeptide corresponding to SEQ ID NO: 1188. SEQ NOS: 578 and 1188 are fragments of SEQ ID NO: 1 and have 100% sequence identity to portions of SEQ ID NO: 1. In some embodiments, a spHT complementary pair comprises a peptide having 100% sequence identity to SEQ ID NO: 578; such a peptide is referred to herein as “SmHT.” In some embodiments, a spHT complementary pair comprises a polypeptide having 100% sequence identity to SEQ ID NO: 1188; such a polypeptide is referred to herein as “LgHT.” Extensive experiments have been conducted to analyze variants of SmHT and LgHT. SEQ ID NOS: 579-1187 correspond to peptide variants having at least one and up to all positions of SEQ ID NO: 588 substituted. A peptide of each of SEQ ID NOS: 578-1187 was synthesized and tested for various characteristics, including the ability to form an active complex with a complementary LgHT variant polypeptide. SEQ ID NOS: 1189-3033 correspond to polypeptide variants having one or more substitutions relative to SEQ ID NO: 1188. A polypeptide of each of SEQ ID NOS: 1188-3033 was synthesized and tested for various characteristics, including the ability to form an active complex with a complementary SmHT variant peptide.

In some embodiments, provided herein is a SmHT peptide or SmHT variant peptide (e.g., fused to a dsRNA binding domain) having at least 70% (e.g., 70%, 75%, 80%, 85%, 90%, 95%, 100%, or ranges therebetween) sequence similarity (e.g., conservative or semi-conservative similarity) with one of SEQ ID NOS: 578-1187. In some embodiments, a peptide (e.g., fused to a dsRNA binding domain) corresponds to SmHT (SEQ ID NO: 578), but with one or more (e.g., 2, 3, 4, 5, 6, 7, 8, 9, 10, 11, 12, 13, 14, 15, 16, 17, or ranges therebetween) of the substitutions of one or more of SEQ ID NOS: 588-1187 relative to SEQ ID NO: 578. In some embodiments, a SmHT variant (e.g., fused to a dsRNA binding domain) has 1-8 (e.g., 1, 2, 3, 4, 5, 6, 7, 8, or ranges therebetween) non-conservative substitutions relative to one of SEQ ID NOS: 578-1187. In some embodiments, provided herein is a SmHT peptide or SmHT variant peptide (e.g., fused to a dsRNA binding domain) comprising:
X₁ X₂ X₃ X₄X₅ (F/W/Y/M/H) X₇ (F/W/Y/D/R) X₉X₁₀X₁₁ (F/W/Y/M/H/R) (V/I/L/M/A/C)
X₁₄ (V/I/L/A/C/MI/L/F/W) X₁₆ X₁₇ (SEQ ID NO: 3034); and/or
X₁ X₂ X₃ X₄ X₅ (F/W/Y) X₇ (F/W/Y) X₉X₁₀X₁₁ (F/W/Y) (V/I/L/M) X₁₄ (V/I/L) X₁₆X₁₇ (SEQ ID NO: 3035);
   wherein each X is any amino acid (e.g., proteinogenic amino acid).

In some embodiments, provided herein is a LgHT polypeptide or LgHT variant polypeptide (e.g., fused to a dsRNA binding domain) having at least 70% (e.g., 70%, 75%, 80%, 85%, 90%, 95%, 100%, or ranges therebetween) sequence similarity (e.g., conservative or semi-conservative similarity) with one of SEQ ID NOS: 1188-3033. In some embodiments, a polypeptide (e.g., within a fusion herein or as a standalone reporter or tag, etc.) corresponds to LgHT (SEQ ID NO: 1188), but with one or more (e.g., 2, 3, 4, 5, 6, 7, 8, 9, 10, 11, 12, 13, 14, 15, 16, 17, 18, 19, 20, 25, 30, or more, or ranges therebetween) of the substitutions of one or more of SEQ ID NOS: 1189-3033 relative to SEQ ID NO: 1188. In some embodiments, a LgHT variant (e.g., fused to a dsRNA binding domain) has at least 70% (e.g., 70%, 75%, 80%, 85%, 90%, 95%, 100%, or ranges therebetween) sequence identity with one of SEQ ID NOS: 1188-3033.

### B. NanoLuc

In some embodiments, provided herein are fusions of dsRNA bounding domains with complementary pairs of peptides/polypeptides capable of interacting with each other (e.g., facilitated by binding of dsRNA binding domains to a dsRNA) to form a luminescent complex capable of interacting with a luminescent substrate to produce luminescence. In some embodiments, the luminescent complex is capable of producing significantly enhanced luminescence upon interaction with the luminescent substrate than either of the complementary peptides/polypeptides alone or in the presence of the luminescent substrate without the other peptide/polypeptide of the complementary pair.

In some embodiments, a first fusion is provided comprising a first complementary peptide or polypeptide fragment of a luciferase, and a second fusion is provided comprising a second complementary peptide or polypeptide fragment of the luciferase, wherein upon interacting (e.g., facilitated by binding of the dsRNA bounding domains to a dsRNA), the complementary peptide(s)/polypeptide(s) form an active luciferase complex capable generating luminescence in the presence (upon contacting) a suitable substrate for the luciferase. In some embodiments, the complementary peptide(s)/polypeptide(s) are fragments of a split luciferase.

In some embodiments, the peptide/polypeptide components capable of forming a luciferase complex are fragments of a split luciferase, such as those derived from the commercially available NANOLUC luciferase (Promega), including but not limited to the NANOBIT (Promega) SMBIT peptide (Promega) and the LGBIT polypeptide (Promega), which are capable of facilitated formation of a luminescent complex. In some embodiments, the peptide/polypeptide components capable of forming a luciferase complex are any of the peptide and polypeptide components described in U.S. Pat. No. 9,797,889 and/or U.S. application Ser. No. 16/439,565 (incorporated by reference in their entireties).

In some embodiments, provided herein are compositions (e.g., fusion polypeptides) and systems (e.g., multiple complementary fusion polypeptides, substrates, etc.) comprising peptide/polypeptide fragments capable of interacting (e.g., facilitated by binding of dsRNA bounding domains fused thereto to a dsRNA) to form an active luminescent complex capable of utilizing an appropriate substrate to generate luminescence.

In some embodiments, provided herein are fusion polypeptides and systems thereof (e.g., multiple complementary fusion polypeptides, substrates, etc.) comprising dsRNA bounding domains fused to complementary peptide/polypeptide fragments capable of interacting (e.g., facilitated by binding of dsRNA bounding domains fused thereto to a dsRNA) to form an active bioluminescent complex capable of generating luminescence upon interaction with an appropriate luminescent substrate. In some embodiments, a first fusion is provided comprising a peptide/polypeptide fragment of a luminescent protein, and a second fusion is provided comprising a complementary peptide/polypeptide fragment of the luminescent protein, wherein upon interacting (e.g., facilitated by binding of dsRNA bounding domains fused thereto to a dsRNA), the complementary peptide/polypeptide fragments form an active bioluminescent complex capable of generating luminescence upon interaction with an appropriate luminescent substrate. In some embodiments, the complementary peptide(s)/polypeptide(s) are fragments of a split luminescent protein (e.g., luciferase).

In some embodiments, provided herein, are pairs of fusions of dsRNA binding domains with the components of a binary complementation system capable of forming a luminescent complex. In other embodiments, a tertiary or multiplex complementation system (e.g., 3 or more components) finds use in the fusions with dsRNA binding domains and systems herein. For example, fusions of dsRNA binding domains with three or more components of a system may be provided. Alternatively, a system may comprise two fusions of dsRNA binding domains with components of a luminescent complex, and one or more additional components of the luminescent complex as isolated components.

The native Oplophorus gracilirostris luciferase (OgLuc) and commercially-available NANOLUC luciferase (Promega Corporation) each comprise polypeptides of 10 β (beta) strands (β1, β2, β3, β4, β5, β6, β7, β8, β9, β10). U.S. Pat. No. 9,797,889 (herein incorporated by reference in its entirety) describes development and use of a complementation system comprising a β1-9-like polypeptide and a β10-like peptide (certain OgLuc/NANOLUC-based polypeptide and peptide sequences in polypeptide and peptide sequences in U.S. Pat. No. 9,797,889 differ from the corresponding sequences in NANOLUC and wild-type native OgLuc). Similarly, U.S. application Ser. No. 16/439,565 (herein incorporated by reference in its entirety) describes the development and use of a complementation systems comprising two or more OgLuc/NANOLUC peptides and/or polypeptides (certain OgLuc/NANOLUC-based polypeptide and peptide sequences in U.S. patent Ser. No. 16/439,565 differ from the corresponding sequences in NANOLUC and wild-type native OgLuc).

In some embodiments, provided in the fusions with dsRNA binding domains herein is a peptide component of a binary bioluminescent complex having greater than 40% (e.g., >40%, >45%, >50%, >55%, >60%, >65%, >70%, >75%, >80%, >85%, >90%, >95%, >98%, >99%, 100%) sequence identity with SEQ ID NO: 3036, wherein a detectable bioluminescent signal is produced when the peptide component of a binary bioluminescent complex contacts a polypeptide consisting of SEQ ID NO: 3037 (e.g., facilitated by binding of the dsRNA binding domain to a dsRNA) in the presence of a substrate for the bioluminescent complex (e.g., greater luminescence that the components of the complex in the presence of the substrate). In some embodiments, the peptide (e.g., within a dsRNA binding domain fusion) has less than 100% sequence identity with SEQ ID NO: 3036. In some embodiments, a detectable bioluminescent signal is produced when the peptide component of a binary bioluminescent complex contacts (e.g., facilitated by binding of dsRNA binding domains to a dsRNA) a polypeptide component of the binary bioluminescent complex having greater than 40% (e.g., >40%, >45%, >50%, >55%, >60%, >65%, >70%, >75%, >80%, >85%, >90%, >95%, >98%, >99%, 100%) sequence identity with SEQ ID NO: 3037. In certain embodiments, the detectable bioluminescent signal is produced, or is substantially increased, when the peptide associates with the polypeptide comprising or consisting of SEQ ID NO: 3037. In preferred embodiments, the peptide exhibits alteration (e.g., enhancement) of one or more traits compared to a peptide of SEQ ID NO: 3038 or 3039, wherein the traits are selected from: affinity for the polypeptide consisting of SEQ ID NO: 3037, expression, intracellular solubility, intracellular stability, and bioluminescent activity when combined with the polypeptide consisting of SEQ ID NO: 3037 (e.g., within the context of the dsRNA binding domain fusions herein).

Exemplary sequences of peptide components of binary bioluminescent complexes that find use in the embodiments herein (e.g., as portions of fusions with dsRNA binding domain fusions) are described, for example, in U.S. Pat. No. 9,797,889 (incorporated by reference in its entirety). Although a peptide component of binary bioluminescent complexes (e.g., within fusions herein) are not limited to these sequences, in some embodiments, a peptide component of a binary bioluminescent complex herein may be selected from amino acid sequences of SEQ ID NOS: 3-438 and 2162-2365 of U.S. Pat. No. 9,797,889 (incorporated by reference in its entirety).

In some embodiments, provided herein is a peptide component of a binary bioluminescent complex (e.g., as a fusion with a dsRNA binding domain) having greater than 40% (e.g., >40%, >45%, >50%, >55%, >60%, >65%, >70%, >75%, >80%, >85%, >90%, >95%, >98%, >99%, 100%) sequence identity with SEQ ID NO: 3038, wherein a detectable bioluminescent signal is produced when the peptide component of the binary bioluminescent complex contacts (e.g., facilitated by binding of a dsRNA binding domain fused thereto to a dsRNA) a polypeptide consisting of SEQ ID NO: 3037 (e.g., fused to a dsRNA binding domain) in the presence of a substrate for the bioluminescent complex (e.g., greater luminescence that the components of the complex in the presence of the substrate). In some embodiments, the peptide has less than 100% sequence identity with SEQ ID NO: 3036. In some embodiments, a detectable bioluminescent signal is produced when the peptide component of a binary bioluminescent complex (e.g., fused to a dsRNA binding domain) contacts a polypeptide component of the binary bioluminescent complex (e.g., fused to a dsRNA binding domain) having greater than 40% (e.g., >40%, >45%, >50%, >55%, >60%, >65%, >70%, >75%, >80%, >85%, >90%, >95%, >98%, >99%, 100%) sequence identity with SEQ ID NO: 3037. In certain embodiments, the detectable bioluminescent signal is produced, or is substantially increased, when the peptide associates with the polypeptide comprising or consisting of SEQ ID NO: 3037.

Exemplary sequences of polypeptide components of binary bioluminescent complexes that find use in the embodiments herein (e.g., as fusions with a dsRNA binding domain) are described, for example, in U.S. Pat. No. 9,797,889 (incorporated by reference in its entirety). Although the peptide components of binary bioluminescent complexes herein are not limited to these sequences, in some embodiments, the polypeptide component of a binary bioluminescent complex herein may be selected from amino acid sequences of SEQ ID NOS: 441-2156 of U.S. Pat. No. 9,797,889 (incorporated by reference in its entirety).

In some embodiments, provided herein is a fusion of a dsRNA binding domain and a polypeptide component of a binary bioluminescent complex having greater than 40% (e.g., >40%, >45%, >50%, >55%, >60%, >65%, >70%, >75%, >80%, >85%, >90%, >95%, >98%, >99%, 100%) sequence identity with SEQ ID NO: 3037, wherein a detectable bioluminescent signal is produced when the polypeptide contacts a peptide consisting of SEQ ID NO: 3036 or 3038 (e.g., within a fusion herein) in the presence of a substrate for the bioluminescent complex (e.g., greater luminescence that the components of the complex in the presence of the substrate).

In some embodiments, provided herein are pairs of fusions, each comprising a dsRNA binding domain and the first fusion comprising a first component of a bioluminescent complex having 40% or greater (e.g., 40%, 45%, 50%, 55%, 60%, 65%, 70%, 75%, 80%, 85%, 90%, 95%, or more (e.g., 100%), or ranges therebetween) sequence identity to a first fragment of SEQ ID NO: 3041 and the second fusion comprising a second component of the bioluminescent complex comprising 40% or greater (e.g., 40%, 45%, 50%, 55%, 60%, 65%, 70%, 75%, 80%, 85%, 90%, 95%, or more (e.g., 100%), or ranges therebetween) sequence identity to the complementary portion of SEQ ID NO: 3041. In some embodiments, the first component comprises 40% or greater (e.g., 40%, 45%, 50%, 55%, 60%, 65%, 70%, 75%, 80%, 85%, 90%, 95%, or more (e.g., 100%), or ranges therebetween) sequence identity to SEQ ID NO: 3042, and the complementary component comprises 40% or greater (e.g., 40%, 45%, 50%, 55%, 60%, 65%, 70%, 75%, 80%, 85%, 90%, 95%, or more (e.g., 100%), or ranges therebetween) sequence identity to SEQ ID NO: 3050. In some embodiments, the first component comprises 40% or greater (e.g., 40%, 45%, 50%, 55%, 60%, 65%, 70%, 75%, 80%, 85%, 90%, 95%, or more (e.g., 100%), or ranges therebetween) sequence identity to SEQ ID NO: 3043, and the complementary component comprises 40% or greater (e.g., 40%, 45%, 50%, 55%, 60%, 65%, 70%, 75%, 80%, 85%, 90%, 95%, or more (e.g., 100%), or ranges therebetween) sequence identity to SEQ ID NO: 3051. In some embodiments, the first component comprises 40% or greater (e.g., 40%, 45%, 50%, 55%, 60%, 65%, 70%, 75%, 80%, 85%, 90%, 95%, or more (e.g., 100%), or ranges therebetween) sequence identity to SEQ ID NO: 3044, and the complementary component comprises 40% or greater (e.g., 40%, 45%, 50%, 55%, 60%, 65%, 70%, 75%, 80%, 85%, 90%, 95%, or more (e.g., 100%), or ranges therebetween) sequence identity to SEQ ID NO: 3052. In some embodiments, the first component comprises 40% or greater (e.g., 40%, 45%, 50%, 55%, 60%, 65%, 70%, 75%, 80%, 85%, 90%, 95%, or more (e.g., 100%), or ranges therebetween) sequence identity to SEQ ID NO: 3045, and the complementary component comprises 40% or greater (e.g., 40%, 45%, 50%, 55%, 60%, 65%, 70%, 75%, 80%, 85%, 90%, 95%, or more (e.g., 100%), or ranges therebetween) sequence identity to SEQ ID NO: 3053. In some embodiments, the bioluminescent signal is substantially increased when the first component associates with the complementary component (e.g., facilitated by the fused dsRNA binding domains binding to a dsRNA).

Exemplary sequences of peptide and polypeptide components of binary or multipartite bioluminescent complexes that find use in the fusions with dsRNA binding domains in embodiments herein are described, for example, in U.S. application Ser. No. 16/439,565 (incorporated by reference in its entirety). Although the peptide and polypeptide components of binary or multipartite bioluminescent complexes herein are not limited to these sequences, in some embodiments, a peptide or polypeptide component of a binary or multipartite bioluminescent complex herein may be selected from amino acid sequences of SEQ ID NOS: 1-804 of U.S. application Ser. No. 16/439,565 (incorporated by reference in its entirety).

In some embodiments, provided herein is a β6-7-like peptide (e.g., within a fusion described herein) comprising SEQ ID NOS: 3054 and 3055. In some embodiments, provided herein is a β6-7-like peptide having 40% or greater (e.g., 40%, 45%, 50%, 55%, 60%, 65%, 70%, 75%, 80%, 85%, 90%, 95%, or more (e.g., 100%), or ranges therebetween) sequence identity with SEQ ID NOS: 3054 and 3055.

In some embodiments, provided herein is a β7-8-like peptide (e.g., within a fusion described herein) comprising SEQ ID NOS: 3055 and 3056. In some embodiments, provided herein is a β7-8-like peptide (e.g., within a fusion described herein) having 40% or greater (e.g., 40%, 45%, 50%, 55%, 60%, 65%, 70%, 75%, 80%, 85%, 90%, 95%, or more (e.g., 100%), or ranges therebetween) sequence identity with SEQ ID NOS: 3055 and 3056.

In some embodiments, provided herein is a β8-9-like peptide (e.g., within a fusion described herein) comprising SEQ ID NOS: 3056/3059 or 3056/3060. In some embodiments, provided herein is a β8-9-like peptide (e.g., within a fusion described herein) having 40% or greater (e.g., 40%, 45%, 50%, 55%, 60%, 65%, 70%, 75%, 80%, 85%, 90%, 95%, or more (e.g., 100%), or ranges therebetween) sequence identity with NOS: 3056/3059 or 3056/3060.

In some embodiments, provided herein is a β9-10-like peptide (e.g., within a fusion described herein) comprising SEQ ID NOS: 3059/3057, 3059/3058, 3060/3057, or 3060/3058. In some embodiments, provided herein is a β8-9-like peptide (e.g., within a fusion described herein) having 40% or greater (e.g., 40%, 45%, 50%, 55%, 60%, 65%, 70%, 75%, 80%, 85%, 90%, 95%, or more (e.g., 100%), or ranges therebetween) sequence identity with NOS: SEQ ID NOS: 3059/3057, 3059/3058, 3060/3057, or 3060/3058.

In some embodiments, provided herein is a β6-8-like peptide or polypeptide (e.g., within a fusion described herein) comprising SEQ ID NOS: 3054-3056. In some embodiments, provided herein is a β6-8-like peptide or polypeptide (e.g., within a fusion described herein) having 40% or greater (e.g., 40%, 45%, 50%, 55%, 60%, 65%, 70%, 75%, 80%, 85%, 90%, 95%, or more (e.g., 100%), or ranges therebetween) sequence identity with NOS: SEQ ID NOS: 3054-3056.

In some embodiments, provided herein is a β7-9-like peptide or polypeptide (e.g., within a fusion described herein) comprising SEQ ID NOS: 3055/3056/3059 or 3055/3056/3060. In some embodiments, provided herein is a β7-9-like peptide or polypeptide (e.g., within a fusion described herein) having 40% or greater (e.g., 40%, 45%, 50%, 55%, 60%, 65%, 70%, 75%, 80%, 85%, 90%, 95%, or more (e.g., 100%), or ranges therebetween) sequence identity with NOS: SEQ ID NOS: 3055/3056/3059 or 3055/3056/3060.

In some embodiments, provided herein is a β8-10-like peptide or polypeptide (e.g., within a fusion described herein) comprising SEQ ID NOS: 3056/3059/3057, 3056/3059/3058, 3056/3060/3057, or 3056/3060/3058. In some embodiments, provided herein is a β7-9-like peptide or polypeptide (e.g., within a fusion described herein) having 40% or greater (e.g., 40%, 45%, 50%, 55%, 60%, 65%, 70%, 75%, 80%, 85%, 90%, 95%, or more (e.g., 100%), or ranges therebetween) sequence identity with NOS: SEQ ID NOS: 3056/3059/3057, 3056/3059/3058, 3056/3060/3057, or 3056/3060/3058.

### C. Fluorescent Protein

In some embodiments, provided herein are fusions of dsRNA bounding domains with complementary pairs of peptides/polypeptides capable of interacting with each other (e.g., facilitated by binding of dsRNA binding domains to a dsRNA) to form a fluorescent complex capable emitting fluorescent light within a detectable range (emission spectra) upon excitation with appropriate wavelength(s) (excitation spectra). In some embodiments, the fluorescent complex is capable of producing significantly enhanced fluorescence than either of the complementary peptides/polypeptides alone.

In some embodiments, a first fusion is provided comprising a first complementary peptide or polypeptide fragment of a fluorescent protein, and a second fusion is provided comprising a second complementary peptide or polypeptide fragment of the fluorescent protein, wherein upon interacting (e.g., facilitated by binding of the dsRNA bounding domains to a dsRNA), the complementary peptide(s)/polypeptide(s) form an active fluorescent complex capable of emitting fluorescent light within a detectable range (emission spectra) upon excitation with appropriate wavelength(s) (excitation spectra). In some embodiments, the complementary peptide(s)/polypeptide(s) are fragments of a fluorescent protein.

In some embodiments, fragments of a fluorescent protein are provided for use in embodiments herein. Exemplary fluorescent proteins include, but are not limited to yellow fluorescent protein (YFP), green fluorescent protein (GFP), cyan fluorescent protein (CFP), red fluorescent protein (RFP), umbelliferone, fluorescein, fluorescein isothiocyanate, rhodamine, dichlorotriazinylamine fluorescein, cyanines, dansyl chloride, phycocyanin, and phycoerythrin. Table 1 provides examples of existing split fluorescent proteins that may find use (e.g., with the two components fused to dsRNA bounding domains) in embodiments herein.

TABLE 1
Exemplary split fluorescent proteins

| | | Citation (incorporated by |
|---|---|---|
| Fluorescent Protein | Description | reference in their entireties) |
| EBFP2 | EBFP2(1-10) and | Multiplexed labeling of cellular proteins with |
| | Capri(1-10) for use with | split fluorescent protein tags. Tamura R, Jiang |
| | GFP(11) | F, Xie J, Kamiyama D. Commun Biol. 2021 |
| Cerulean | Cerulean(1-10) for use | Multiplexed labeling of cellular proteins with |
| | with GFP(11) | split fluorescent protein tags. Tamura R, Jiang |
| | | F, Xie J, Kamiyama D. Commun Biol. 2021 |
| ECFP | C155 of ECFP pairs with | Identification of new fluorescent protein |
| | N173 derived from | fragments for bimolecular fluorescence |
| | Cerulean or Venus | complementation analysis under physiological |
| | | conditions. Shyu Y J, Liu H, Deng X, Hu C D. |
| | | Biotechniques. 2006 |
| sfGFP | Split super-folder GFP, | Versatile protein tagging in cells with split |
| | our most-requested | fluorescent protein. Kamiyama et al. Nat |
| | GFP(1-10) and GFP(11) | Commun. 2016 |
| spGFP | Split superpositive GFP | Split-superpositive GFP reassembly is a fast, |
| | | efficient, and robust method for detecting |
| | | protein-protein interactions in vivo. Blakeley |
| | | B D, Chapman A M, McNaughton B R. Mol |
| | | Biosyst. 2012 |
| mNeonGreen2 | mNG2(1-10) and | Improved split fluorescent proteins for |
| | mNG2(11) | endogenous protein labeling. Feng et al. Nat |
| | | Commun. 2017 |
| mNeonGreen3 | mNG3K(1-10) and | Improved yellow-green split fluorescent |
| | mNG3A(1-10) for use | proteins for protein labeling and signal |
| | with mNG2(11) | amplification. Zhou S, Feng S, Brown D, |
| | | Huang B. PLoS One. 2020 |
| Venus | pBiFC-VN173, pBiFC- | Identification of new fluorescent protein |
| | VC155 | fragments for bimolecular fluorescence |
| | | complementation analysis under physiological |
| | | conditions. Shyu Y J, Liu H, Deng X, Hu C D. |
| | | Biotechniques. 2006 |
| Venus | Improved N-terminal | An improved bimolecular fluorescence |
| | fragment VN155(I152L), | complementation assay with a high signal-to- |
| | for use with VC155 | noise ratio. Kodama Y, Hu C D. |
| | | Biotechniques. 2010 |
| mVenus | pET-BiFC contains both | An enhanced recombinant amino-terminal |
| | fragments of split | acetylation system and novel in vivo high- |
| | mVenus (aa 155), | throughput screen for molecules affecting |
| | includes I152L in N- | alpha-synuclein oligomerisation. Eastwood T, |
| | terminal fragment | Baker K, Brooker H, Frank S, Mulvihill D P. |
| | | FEBS Lett. 2017 |
| mVenus | Split mVenus (aa 155), | Constant rate of p53 tetramerization in |
| | includes I152L in N- | response to DNA damage controls the p53 |
| | terminal fragment | response. Gaglia G, Lahav G. Mol Syst Biol. |
| | | 2014 |
| EYFP | Gateway vectors with | Arabidopsis homolog of the yeast TREX-2 |
| | EYFP split at 175 | mRNA export complex: components and |
| | | anchoring nucleoporin. Lu et al. Plant J. 2010 |
| mScarlet | Split-wrmScarlet variant | Split-wrmScarlet and split-sfGFP: tools for |
| | of mScarlet | faster, easier fluorescent labeling of |
| | | endogenous proteins in Caenorhabditis |
| | | elegans. Goudeau et al. Genetics. 2021 |
| mRuby4 | mRuby4(1-10), pairs with | Multiplexed labeling of cellular proteins with |
| | sfCherry2(11) | split fluorescent protein tags. Tamura R, Jiang |
| | | F, Xie J, Kamiyama D. Commun Biol. 2021 |
| sfCherry2 | sfCherry2(1-10) and | Improved split fluorescent proteins for |
| | sfCherry2(11), also | endogenous protein labeling. Feng et al. Nat |
| | photo-activatable variant | Commun. 2017 |
| | PAsfCherry2(1-10) | |
| sfCherry3 | Improved sfCherry3C(1- | Bright split red fluorescent proteins for the |
| | 10) for use with | visualization of endogenous proteins and |
| | sfCherry2(11) | synapses. Feng S et al. Commun Biol. 2019 |
| iRFP | iSplit, comprised of PAS | A Near-Infrared BiFC Reporter for In Vivo |
| | and GAF domains of | Imaging of Protein-Protein Interactions. |
| | iRFP713. Requires | Filonov G S, Verkhusha V V. Chem Biol. 2013 |
| | presence of heme | |
| | oxygenase for | |
| | chromophore formation | |
| | (abundant in eukaryotes) | |
| iRFP | GAF domains from | Bright monomeric near-infrared fluorescent |
| | miRFP709 and | proteins as tags and biosensors for multiscale |
| | miRFP670, pair with | imaging. Shcherbakova et al. Nat Commun. |
| | common PAS domain. | 2016 |
| | Requires presence of | |
| | heme oxygenase for | |
| | chromophore formation | |
| | (abundant in eukaryotes) | |
| FAST | Fluorescence-activating | A split fluorescent reporter with rapid and |
| | and absorption shifting | reversible complementation. Tebo A G, |
| | tag (FAST) for use with | Gautier A. Nat Commun. 2019 |
| | green-yellow or orange- | |
| | red fluorogen | |

As an exemplary embodiment, components of a split GFP (a peptide of SEQ ID NO: 3070 and a polypeptide of SEQ ID NO: 3068) were fused to dsRNA binding domains (SEQ ID NO: 3061) by linkers of SEQ ID NO: 3064. In some embodiments, provided herein are pairs of fusions, each comprising a dsRNA binding domain and the first fusion comprising a first component of a fluorescent complex having 40% or greater (e.g., 40%, 45%, 50%, 55%, 60%, 65%, 70%, 75%, 80%, 85%, 90%, 95%, or more (e.g., 100%), or ranges therebetween) sequence identity to a first fragment of SEQ ID NO: 3068 and the second fusion comprising a second component of the bioluminescent complex comprising 40% or greater (e.g., 40%, 45%, 50%, 55%, 60%, 65%, 70%, 75%, 80%, 85%, 90%, 95%, or more (e.g., 100%), or ranges therebetween) sequence identity to the complementary portion of SEQ ID NO: 3070.

### III. Fusions

In some embodiments, the fusions herein comprise a dsRNA binding domain directly linked to a component of a detectable complex. In some embodiments, a dsRNA binding domain comprises two directly linked dsRNA binding motifs. However, in other embodiments, the dsRNA binding domain and component of the detectable complex, and/or two dsRNA binding motifs in a dsRNA binding domain are fused via a linker. Such linkers may be of any suitable sequence and up to 100 amino acids in length (e.g., 1, 2, 3, 4, 5, 6, 7, 8, 9, 10, 15, 20, 25, 30, 35, 40, 445, 50, 60, 70, 80, 90, 100, or ranges therebetween). In some embodiments, the fusions herein comprise linkers to facilitate optimized geometries for complementation, ligand/substrate binding, dsRNA binding, etc.

In some embodiments, the components (e.g., dsRNA binding domain and/or motifs, detectable complex components, linkers, etc.) of a fusion herein may be arranged in any suitable orientation that allows for dsRNA binding, structural complementation to for a detectable complex, and detectable signal/activity from the detectable complex. In some embodiments, the C-terminus of a dsRNA binding domain is fused (e.g., directly or via a linker) to the N-terminus of a component of a detectable complex. In some embodiments, the N-terminus of a dsRNA binding domain is fused (e.g., directly or via a linker) to the C-terminus of a component of a detectable complex. In some embodiments, the C-terminus of a first dsRNA binding motif is fused (e.g., directly or via a linker) to the N-terminus of a component of a detectable complex, and the N-terminus of a second dsRNA binding motif is fused (e.g., directly or via a linker) to the C-terminus of the component of the detectable complex.

### IV. Substrates, Ligands, and Cofactors

In some embodiments, the detectable complexes that find use in embodiments herein utilize a substrate, ligand, cofactor, etc. to produce a detectable signal. In such embodiments, systems and methods are provided comprising the appropriate substrate, cofactor, and/or ligand for the detectable complex and components thereof.

In some embodiments, ligands/substrates of the invention are permeable to the plasma membranes of cells (i.e., capable of passing from the exterior of a cell (e.g., eukaryotic, prokaryotic) to the cellular interior without chemical, enzymatic, or mechanical disruption of the cell membrane).

In some embodiments, ligands herein comprise a cleavable linker, for example, those described in U.S. Pat. No. 10,618,907; incorporated by reference in its entirety.

For systems and methods herein utilizing various split enzymes as detectable complexes, the appropriate substrate and/or cofactor is provided for use with such systems. For example, dihydrofolate, ATP, tetramethylbenzidine, chorismite, etc. may be provided to generate a detectable signal with the assembled detectable complex.

### A. Haloalkyl Ligands

In embodiments in which a detectable complex comprises a modified dehalogenase complex (e.g., a split HALOTAG), systems and methods are provided comprising a haloalkane ligand. The modified dehalogenase complex (e.g., formed by structural complementation facilitated by binding of the fusions herein to dsRNA) binds the haloalkane ligand and a functional group linked to the haloalkane allows for detection. In some embodiments, the haloalkane ligand is of formula (I): R-linker-A-X, wherein R is a detectable functional group, wherein the linker is a multiatom straight or branched chain including C, N, S, or O, or a group that comprises one or more rings, e.g., saturated or unsaturated rings, such as one or more aryl rings, heteroaryl rings, or any combination thereof, wherein A-X is a ligand for a modified dehalogenase (e.g., HALOTAG) (e.g., wherein A is (CH₂)₄₋₂₀ and X is a halide (e.g., Cl or Br)). Suitable ligands are described, for example, in U.S. Pat. Nos. 11,072,812; 11,028,424; 10,618,907; and 10,101,332; incorporated by reference in their entireties. In certain embodiments, X of formula (I) is a methylsulfonamide or trifluoromethylsulfonamide, rather than a halide; such an embodiment results in an exchangeable ligand that reversibly binds to a modified dehalogenase (e.g., HALOTAG). Such ligands are described in, for example, Kompa et al. J. Am. Chem. Soc. 2023, 145, 5, 3075-3083; incorporated by reference in its entirety.

In some embodiments, R is one or more functional groups (such as a fluorophore, biotin, luminophore, or a fluorogenic or luminogenic molecule). Exemplary functional groups for use in the invention include, but are not limited to, an amino acid, protein, e.g., enzyme, antibody or other immunogenic protein, a radionuclide, a nucleic acid molecule, a drug, a lipid, biotin, avidin, streptavidin, a magnetic bead, a solid support, an electron opaque molecule, chromophore, MRI contrast agent, a dye, e.g., a xanthene dye, a calcium sensitive dye, e.g., 1-[2-amino-5-(2,7-dichloro-6-hydroxy-3-oxy-9-xanthenyl)-phenoxy]-2-(2′-am-ino-5′-methylphenoxy) ethane-N,N,N′,N′-tetraacetic acid (Fluo-3), a sodium sensitive dye, e.g., 1,3-benzenedicarboxylic acid, 4,4′-[1,4,10,13-tetraoxa-7,16-diazacyclooctadecane-7,16-diylbis(5-methoxy- -6,2-benzofurandiyl)]bis (PBFI), a NO sensitive dye, e.g., 4-amino-5-methylamino-2′,7′-difluorescein, or other fluorophore. In one embodiment, the functional group is an immunogenic molecule, i.e., one which is bound by antibodies specific for that molecule.

In some embodiments, a ligand comprises a fluorescent functional group (R). Suitable fluorescent functional groups include, but are not limited to: stilbazolium derivatives (Marquesa et al. Mechanism-Based Strategy for Optimizing HaloTag Protein Labeling. ChemRxiv. Cambridge: Cambridge Open Engage; 2021; incorporated by reference in its entirety), xanthene derivatives (e.g., fluorescein, rhodamine, Oregon green, cosin, Texas red, etc.), cyanine derivatives (e.g., cyanine, indocarbocyanine, oxacarbocyanine, thiacarbocyanine, merocyanine, etc.), naphthalene derivatives (e.g., dansyl and prodan derivatives), oxadiazole derivatives (e.g., pyridyloxazole, nitrobenzoxadiazole, benzoxadiazole, etc.), pyrene derivatives (e.g., cascade blue), oxazine derivatives (e.g., Nile red, Nile blue, cresyl violet, oxazine 170, etc.), acridine derivatives (e.g., proflavin, acridine orange, acridine yellow, etc.), arylmethine derivatives (e.g., auramine, crystal violet, malachite green, etc.), tetrapyrrole derivatives (e.g., porphin, phtalocyanine, bilirubin, etc.), CF dye (Biotium), BODIPY (Invitrogen), ALEXA FLOUR (Invitrogen), DYLIGHT FLUOR (Thermo Scientific, Pierce), ATTO and TRACY (Sigma Aldrich), FluoProbes (Interchim), DY and MEGASTOKES (Dyomics), SULFO CY dyes (CYANDYE, LLC), SETAU AND SQUARE DYES (SETA BioMedicals), QUASAR and CAL FLUOR dyes (Biosearch Technologies), SURELIGHT DYES (APC, RPE, PerCP, Phycobilisomes) (Columbia Biosciences), APC, APCXL, RPE, BPE (Phyco-Biotech), autofluorescent proteins (e.g., YFP, RFP, mCherry, mKate), quantum dot nanocrystals, etc.

In some embodiments, a ligand comprises a fluorogenic functional group (R). A fluorogenic functional group is one that produces and enhanced fluorescent signal upon binding of the ligand to a target (e.g., binding of a haloalkane to a modified dehalogenase). By producing significantly increased fluorescence (e.g., 10×, 31×, 50×, 100×, 310×, 500×, 100×, or more) upon target engagement, the problem of background signal is alleviated. Exemplary fluorogenic dyes for use in embodiments herein include the JANELIA FLUOR family of fluorophores, such as:

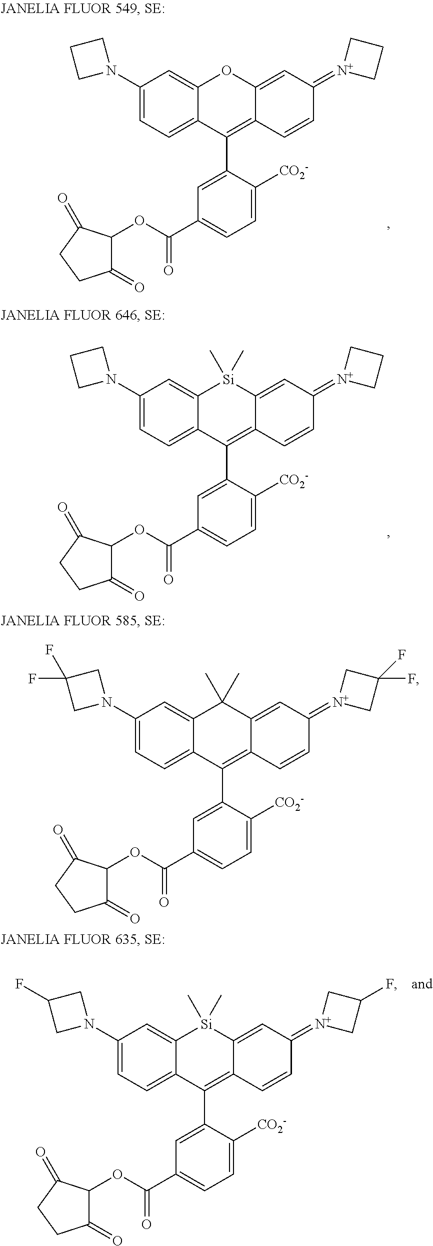

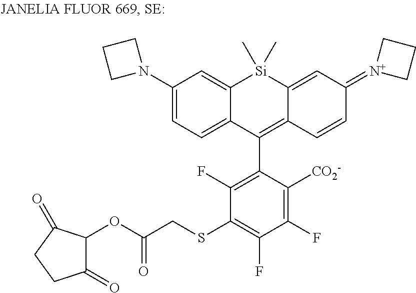

(see, e.g., U.S. Pat. Nos. 9,933,417; 10,018,624; 10,161,932; and 10,495,632; each of which is incorporated by reference in their entireties). In some embodiments, exemplary conjugates of JANELIA FLUOR 549 and JANELIA FLUOR 646 with haloalkane ligands for modified dehalogenase (e.g., HALOTAG) are commercially available (Promega Corp.). The use and design of fluorogenic functional groups, dyes, probes, and ligands is described in, for example, Grimm et al. Nat Methods. 3117 October; 14 (10): 987-994; Wang et al. Nat Chem. 3120 February; 12 (2): 165-172; incorporated by reference in their entireties.

### B. Bioluminescent Substrates

In some embodiments, systems and methods herein comprise a luminescent complex that utilizes a luminophore to generate a detectable luminescent signal. In some embodiments, an appropriate luminophore is selected to pair with the luminescent complex.

In some embodiments, the systems and methods herein (comprising a bioluminescent complex and/or components thereof) utilize an imidazopyrazine luminophore substrate to generate bioluminescence. Such embodiments include those utilizing NANOLUC-based, NANOBIT-based, and NANOTRIP-based luminescent complexes. In some embodiments, the substrate is coelenterazine:

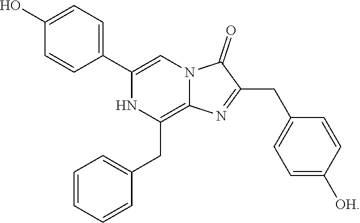 [SMILES: O=c1c(Cc2ccc(O)cc2)nc2c(Cc3ccccc3)[nH]c(-c3ccc(O)cc3)cn1-2]

In some embodiments, the substrate is a coelenterazine derivative, such as, furimazine, furimazine analogs (e.g., fluorofurimazine) coelenterazine-n, coelenterazine-f, coelenterazine-h, coelenterazine-hcp, coelenterazine-cp, coelenterazine-c, coelenterazine-e, coelenterazine-fcp, bis-deoxycoelenterazine (“coelenterazine-hh”), coelenterazine-i, coelenterazine-icp, coelenterazine-v, and 2-methyl coelenterazine, in addition to those disclosed in WO 2003/040100; U.S. application Ser. No. 12/056,073 (paragraph [0086]); U.S. Pat. No. 8,669,103; U.S. Prov. App. No. 63/379,573; the disclosures of which are incorporated by reference herein in their entireties.

In some embodiments, the substrate is furimazine:

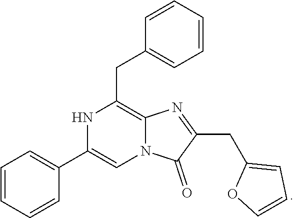 [SMILES: O=c1c(Cc2ccco2)nc2c(Cc3ccccc3)[nH]c(-c3ccccc3)cn1-2]

In some embodiments, the substrate is fluorofurimazine:

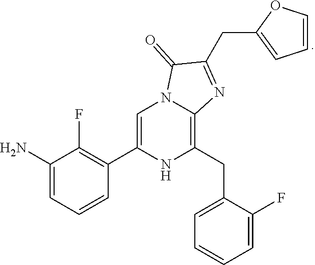 [SMILES: Nc1cccc(-c2cn3c(=O)c(Cc4ccco4)nc-3c(Cc3ccccc3F)[nH]2)c1F]

Suitable luminophores for the bioluminescent complexes used in the systems or methods herein will be understood. For example, firefly luciferin, with the structure:

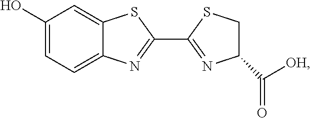 [SMILES: O=C(O)[C@H]1CSC(c2nc3ccc(O)cc3s2)=N1]

is the luciferin found in many Lampyridae species and is the substrate of beetle luciferases. Latia luciferin, with the structure:

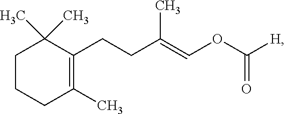

is from the freshwater snail Latia neritoides. Bacterial luciferin, with the structure:

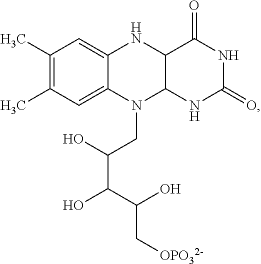

finds use as a substrate for many bacterial luciferases. Coelenterazine, of the structure:

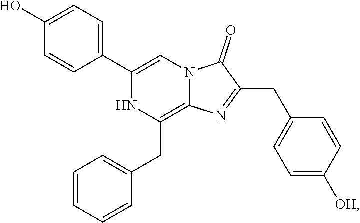 [SMILES: O=c1c(Cc2ccc(O)cc2)nc2c(Cc3ccccc3)[nH]c(-c3ccc(O)cc3)cn1-2]

is found in radiolarians, ctenophores, cnidarians, squid, brittle stars, copepods, chaetognaths, fish, and shrimp and is the luminophore substrate for the luciferases of those organisms. Variants and derivatives of coelenterazine, such as furimazine and fluorofurimazine, find use in embodiments herein (e.g., with Oplophorus-derived bioluminescent complexes). Other luminophore substrates include those of dinoflagellates:

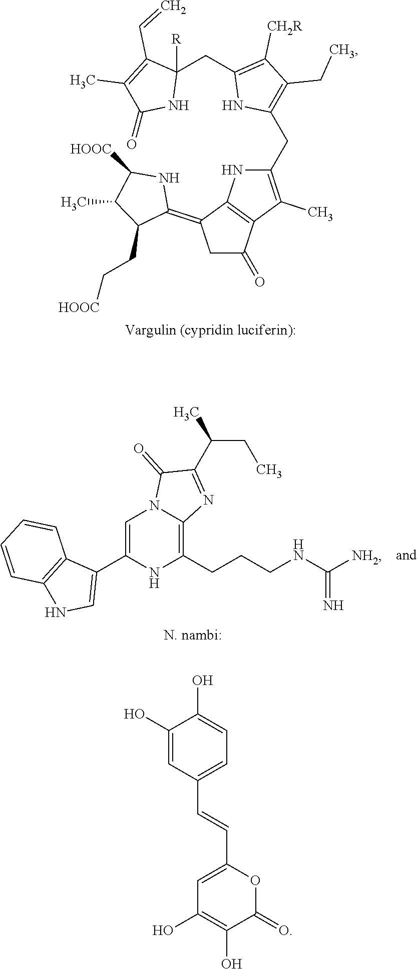

Pairing of appropriate bioluminescent proteins or complexes with luminophores is understood in the field.

### V. Methods

In some embodiments, provided herein are methods of detecting the presence of and/or quantifying the amount of dsRNA is a sample comprising: (a) contacting the sample with a sufficient concentration of a system described herein (e.g., fusions of dsRNA binding domains with a pair of components of a detectable complex) and any necessary substrates, ligands, cofactors, etc.; and (b) detecting and/or quantifying a signal produced by the detectable complex. In some embodiments, the amount of signal (e.g., intensity) is correlated to the amount of dsRNA in the sample. In some embodiments, the signal is compared to a signal from a control sample with a known concentration of dsRNA. In some embodiments, the signal is compared to an established value corresponding to a known concentration of dsRNA in a sample.

In some embodiments, the sample is a biological sample, an environmental sample, a pharmaceutical sample (e.g., containing an RNA (e.g., ssRNA) therapeutic), or any suitable sample type that may contain dsRNA (e.g., as a contaminant). In some embodiments, the sample comprises a therapeutic RNA and the method is performed as quality control testing to ensure a sufficiently low quantity of dsRNA in the sample.

### EXPERIMENTAL

### Example 1

### Split Bioluminescent Protein

Fusions of the PKR dsRNA binding domain with LgBiT and SmBiT (PKR-LgBIT and PKR-SmBIT) were expressed in E. Coli and purified with His tags. The proteins expressed and purified well. Experiments conducted during development of embodiments herein demonstrate that the PKR-LgBiT and PKR-SmBIT components (25 ng/ml each), when added to a sample comprising Poly(I:C) (a synthetic dsRNA analog), along with furimazine, were capable of sensitive (limit of detection <0.1 ng/ml) and rapid (1 hour assay time) dsRNA quantification in an easy to use (add-mix-read) assay format (FIG. 3). The optimal incubation time was determined to be 1 hour. The exemplary assay is quantitative with about 2 logs of linear range.

### Example 2

### Split Fluorescent Protein

Fusions of the PKR dsRNA binding domain with LgGFP and SmGFP (PKR-LgGFP and PKR-SmGFP) were prepared. Experiments conducted during development of embodiments herein demonstrate that the PKR-LgBiT and PKR-SmBiT components, when added to a sample comprising dsRNA and exposed to the excitation wavelength for the GFP, were capable of sensitive and rapid dsRNA quantification (FIG. 4).

### REFERENCES

The following references are herein incorporated by reference in their entireties.
Cheng et al. Visualizing double-stranded RNA distribution and dynamics in living cells by dsRNA binding-dependent fluorescence complementation. Virology. 2015 (PMID: 26351203).
Monsion et al. Efficient detection of long dsRNA in vitro and in vivo using the dsRNA binding domain from FHV B2 protein. Front Plant Sci. 2018. (PMID: 29449856)

1. A double-stranded RNA (dsRNA) detection system comprising:
(a) a first fusion of (i) a first dsRNA binding domain and (ii) a first component of a detectable complex; and
(b) a second fusion of (i) a second dsRNA binding domain and (ii) a second component of the detectable complex;
wherein the first and second components of the detectable complex exhibit low affinity for one another in the absence of facilitation through the binding of the first and second dsRNA binding domains to the dsRNA; and
wherein the first and second components of the detectable complex exhibit low affinity for one another in the absence of facilitation through the binding of the first and second dsRNA binding domains to the dsRNA.

2-10. (canceled)

11. The system of claim 1, wherein the first and second dsRNA binding domains each comprise dsRNA binding motifs having at least 70% sequence identity with SEQ ID NO: 3062 and SEQ ID NO: 3063.

12. The system of claim 11, wherein the first and second dsRNA binding domains each comprise dsRNA binding motifs of SEQ ID NO: 3062 and SEQ ID NO: 3063.

13. (canceled)

14. The system of claim 1, wherein the dsRNA binding domain comprises at least 70% sequence identity with SEQ ID NO: 3061.

15. The system of claim 14, wherein the dsRNA binding domain comprises SEQ ID NO: 3061.

16. The system of claim 1, wherein the detectable complex is capable of generating a signal that can be detected and correlated to the amount of dsRNA in a sample with the system.

17. (canceled)

18. The system of claim 16, wherein the signal comprises one or more of fluorescence, luminescence, enzymatic activity, and ligand binding.

19. The system of claim 1, wherein the first and second components of the detectable complex are fragments of a protein capable of generating a detectable signal, and wherein the detectable complex is capable of generating the detectable signal upon association of the first and second components of the detectable complex.

20-21. (canceled)

22. The system of claim 19, wherein the protein is a fluorescent protein selected from yellow fluorescent protein (YFP), green fluorescent protein (GFP), cyan fluorescent protein (CFP), red fluorescent protein (RFP), umbelliferone, fluorescein, fluorescein isothiocyanate, rhodamine, dichlorotriazinylamine fluorescein, cyanines, dansyl chloride, phycocyanin, and phycoerythrin.

23-24. (canceled)

25. The system of claim 19, wherein the detectable signal is enzymatic activity and the protein is selected from beta-lactamase, dihydrofolate reductase (DHFR), focal adhesion kinase (FAK), Gal4, and horseradish peroxidase.

26-29. (canceled)

30. The system of claim 19, wherein the protein is a luciferase selected from an Oplophorus luciferase, a firefly luciferase, a click beetle luciferase, a Renilla luciferase, cypridina luciferase, an Aequorin photoprotein, and an obelin photoprotein.

31-32. (canceled)

33. The system of claim 1, wherein the first component of the detectable complex comprises at least 70% sequence identity with SEQ ID NO: 3043 and the first component of the detectable complex comprises at least 70% sequence identity with SEQ ID NO: 3051.

34. The system of claim 1, wherein the first component of the detectable complex comprises at least 70% sequence identity with SEQ ID NO: 3044 and the first component of the detectable complex comprises at least 70% sequence identity with SEQ ID NO: 3052.

35. The system of claim 1, wherein the first component of the detectable complex comprises at least 70% sequence identity with SEQ ID NO: 3045 and the first component of the detectable complex comprises at least 70% sequence identity with SEQ ID NO: 3053.

36. The system of claim 30, further comprising a substrate for the luciferase.

37. The system of claim 19, wherein the detectable signal is ligand binding.

38. The system of claim 1, wherein the detectable complex is a modified dehalogenase complex and wherein the first and second components of the modified dehalogenase complex comprise at least 70% sequence identity with first and second fragments of SEQ ID NO: 1.

39. (canceled)

40. The system of claim 38, further comprising a haloalkyl ligand for the modified dehalogenase.

41-42. (canceled)

43. A double-stranded RNA (dsRNA) detection system comprising:
(a) a first fusion of (i) a PKR-derived dsRNA binding domain sequence, and (ii) a peptide component of a bioluminescent complex; and
(b) a second fusion of (i) the PKR-derived dsRNA binding domain sequence, and (ii) a polypeptide component of a bioluminescent complex;
wherein upon binding of the PKR-derived dsRNA binding domain sequences to a dsRNA a luminescent complex is formed by structural complementation of the peptide component and the polypeptide component; and
wherein a luminescent signal produced by the luminescent complex in the presence of the dsRNA and a substrate for the luminescent complex is enhanced when compared to a luminescent signal produced in the absence of the dsRNA.

44-55. (canceled)

56. A method of detecting dsRNA in a sample, the method comprising contacting the sample with a system of claim 1 and detecting a signal from the detectable complex, wherein the amount of signal detected correlates with the amount of dsRNA in the sample.

57. (canceled)
